(19) United States
(12) Patent Application Publication
(10) Pub. No.: US 2026/0159876 A1
(43) Pub. Date: Jun. 11, 2026

(54) TUNABLE FINGERLOOP DNA ANTISENSE MOTIFS FOR NUCLEIC ACID TARGETING AND DETECTION

(71) Applicant: Ohio State Innovation Foundation, Columbus, OH (US)

(72) Inventor: Richard LEASE, New Albany, OH (US)

(21) Appl. No.: 18/706,475

(22) PCT Filed: Nov. 1, 2022
(86) PCT No.: PCT/US2022/079073

§ 371 Date: May 1, 2024

Related U.S. Application Data
(60) Provisional application No. 63/274,125, filed on Nov. 1, 2021.
(60) Provisional application No. 63/281,999, filed on Nov. 22, 2021.

(51) Int. Cl. C12Q 1/6827; C12Q 1/6876

(52) U.S. Cl. CPC C12Q 1/6827; C12Q 1/6876

(57) ABSTRACT

Described herein are detection probes and methods of making and using thereof. The probes can include a nucleic acid sequence including a fingerloop structure. In some embodiments, the fingerloop structure can include: an antisense sequence region located in a loop and in one strand of a stem loop of the fingerloop structure, wherein the antisense sequence region is complementary to a target nucleic acid sequence; and a nonsense sequence region located at the base of the stem loop of the fingerloop structure.

### CROSS-REFERENCE TO RELATED APPLICATIONS

The application claims the benefit of U.S. Provisional Application No. 63/274,125, filed Nov. 1, 2021 and U.S. Provisional Application No. 63/281,999, filed Nov. 22, 2021, which are hereby incorporated herein by reference in their entireties.

### INCORPORATION BY REFERENCE

The content of the XML file named “103361_170WO1.xml” which was created on Nov. 1, 2022, and is 52,759 bytes in size, is hereby incorporated by reference in their entirety.

### BACKGROUND

Fingerloop is a structural antisense sequence motif inspired by the structural antisense components of an E. coli regulatory sRNA, DsrA (Lahiry, et al. 2017 ACS Synth Biol). The antisense sequence, which binds with a target RNA (or DNA) by base-pairing interactions, is sequestered in a single-stranded inverted repeat sequence that forms a stem-loop (also called a hairpin loop). The antisense sequence resides in one strand (or the other) of the stem helix and continues into the loop. Two alternative forms exist: in one (ascending stem), antisense sequence proceeds from the 5′-end and ends in the loop, and in the other (descending stem), the antisense sequence starts in the loop and proceeds down the helical stem to the 3′-end. The opposing strand of the helix is the complement of the antisense strand in the helix stem and serves to form a double helix with the antisense component of the stem. The structural stability of the helix can be varied by altering the length of the helix, increasing the loop size, and introducing mismatches or non-Watson-Crickbase pairs in the stem.

The advantages of fingerloop antisense technology are that the non-antisense strand acts as an intrinsic partner to the antisense strand through the stem helix, increasing the free energy barrier of pairing with a separate, target nucleic acid strand. Together with constraints on the size of the loop sequence at the top of the helix, this structural antisense motif excludes base pairing to targets containing mismatches to nucleotides in the loop and stem region. This configuration of the antisense sequences, which is tunable with respect to intrinsic stability as well as to stability in partnership with its target, will permit detection of nucleic acids while improving the exclusion of false positive or near-match targets in detection technologies that involve base pairing, such as PCR or molecular beacon technology. The structured RNA antisense fingerloops significantly exclude off-target matches when compared to linear RNA probe sequences in vivo as demonstrated in an sRNA-based assay system. Further, this nucleic acid detection technology (a) does not rely on centralization of testing facilities but could be used to form a test similar to a “home pregnancy test” with point-of-care diagnostics, (b) does not rely on enzymes but almost exclusively on nucleic acids derivatives, which makes a fingerloop diagnostic relatively portable and useable at different temperatures.

There is a need for improved detection probes that diminish binding to mismatched target nucleic acids.

The compositions and methods disclosed herein address these and other needs.

### SUMMARY

Described herein are detection probes including a fingerloop structure described herein. Described are also methods for detecting a target nucleic acid sequence using a detection probe comprising a fingerloop structure described herein. Described are also methods for amplifying a nucleic acid sequence using amplification primers comprising a fingerloop structures described herein. Described are also methods for modulating protein expression levels and/or mRNA expression.

The methods of increasing the length of the helix outside the antisense region to “tune” the stability of the fingerloop despite temperature changes. This “tuning” step significantly improves targeting fidelity and decreases false positive results by isolating the nucleic acids interaction from temperature fluctuations that could lead to false positive identifications of targets (by stabilizing mismatches at low temperature).

In some embodiments, described herein are detection probes including: a nucleic acid sequence including a fingerloop structure including: an antisense sequence region located in a loop and in one strand of a stem loop of the fingerloop structure, and a nonsense sequence region located at the base of the stem loop of the fingerloop structure. In some embodiments, the antisense sequence region can be complementary to a target nucleic acid sequence. In some embodiments, the fingerloop structure diminishes base pairing to a mismatched target nucleic acid. In some embodiments, the nucleic acid sequence can further include a fluorescent nucleoside at a 3′ end of an ascending strand or at the 5′ end of a descending strand of the stem loop. In some embodiments, the detection probe has a ΔG of −30 kcal/mol or less.

In some embodiments, described herein are also composition including a detection probe described herein and a pharmaceutically acceptable carrier.

In some embodiments, described herein are methods for detecting a target nucleic acid sequence, the method including: providing a nucleic acid sequence; contacting the nucleic acid sequence with a detection probe described herein; assessing a change in fluorescence intensity. In some embodiments, an increase in fluorescence intensity indicates binding of the detection probe to a target nucleic acid sequence. In some embodiments, the detection probe diminishes base pairing to a mismatched target nucleic acid. In some embodiments, the fingerloop structure provides increased detection specificity of the target nucleic acid sequence.

In some embodiments, described herein are methods of tuning a fingerloop stability including: adding a nonsense nucleic acid sequence to a nucleic acid sequence including a fingerloop structure including: an antisense sequence region located in a loop and in one strand of a stem loop of the fingerloop structure. In some embodiments, the antisense sequence region is complementary to a target nucleic acid sequence. In some embodiments, the nonsense nucleic acid sequence can be located at the base of the stem loop of the fingerloop structure. In some embodiments, the fingerloop stability can be maintained despite temperature changes. In some embodiments, tuning fingerloop stability improves targeting fidelity and/or decrease false positive results.

In some embodiments, described herein are methods for amplifying a nucleic acid sequence including: providing a nucleic acid sample; amplifying a target nucleic acid sequence from the nucleic acid sample using amplification primers in a polymerase chain reaction, wherein the amplification primers include a fingerloop structure including: an antisense sequence region located in a loop and in one strand of a stem loop of the fingerloop structure; and a nonsense sequence region located at the base of the stem loop of the fingerloop structure. In some embodiments, the antisense sequence region can be complementary to the target nucleic acid sequence.

In some embodiments, described herein are methods for modulating protein expression levels and/or mRNA expression levels from at least two target mRNAs in a cell simultaneously, the method including: transforming the cell with a system for measuring the activity of a chimeric deoxyribonucleic acid (DNA), and measuring the protein expression levels and/or mRNA expression levels of the first reporter gene and the second reporter gene.

In some embodiments, the system can include: a chimeric DNA; a first plasmid including a first reporter gene operably linked to a first gene leader sequence; and a second plasmid including a second reporter gene operably linked to a second gene leader sequence. In some embodiments, the chimeric DNA includes a first deoxyribonucleic acid (DNA) sequence operably linked to a second deoxyribonucleic acid (DNA) sequence.

In some embodiments, the first DNA sequence can be present in a first fingerloop structure and the second DNA sequence can be present in a second fingerloop structure. In some embodiments, the first and second fingerloop structures are fingerloop structures described herein. In some embodiments, the target nucleic acid sequence can be an mRNA of the first gene leader sequence. In some embodiments, the target nucleic acid sequence can be an mRNA of the second gene leader sequence.

In some embodiments, the first fingerloop structure can include: an antisense sequence region located in a loop and in one strand of a stem loop of the fingerloop structure; and a nonsense sequence region located at the base of the stem loop of the fingerloop structure. In some embodiments, the antisense sequence region is complementary to an mRNA of the first gene leader sequence. In some embodiments, the second fingerloop structure can include: an antisense sequence region located in a loop and in one strand of a stem loop of the fingerloop structure, and a nonsense sequence region located at the base of the stem loop of the fingerloop structure. In some embodiments, the antisense sequence region can be complementary to an mRNA of the second gene leader sequence. In some embodiments, the nonsense sequence region can be located in the stem loop of the second fingerloop structure. In some embodiments, the first reporter gene encodes a fluorescent protein. In some embodiments, the second reporter gene encodes a fluorescent protein. In some embodiments, the first and second fingerloop structures inhibit the binding of the first and second DNAs to mismatched target sequences.

In some embodiments, the antisense sequence region of the loop can include at least 3 nucleotides. In some embodiments, the antisense sequence region of the stem loop can include at least 6 nucleotides. In some embodiments, the nonsense sequence region of the stem loop comprises at least 1 nucleotide.

### DESCRIPTION OF DRAWINGS

FIGS. 1A-1B show an illustration of (1A) the two configurations (ascending or descending) for a fingerloop structure including antisense sequence region and a nonsense sequence and (1B) complex formation between the DNA target variant of hydA mRNA leader (hydA+45mer oligo) and two different fingerloops hydA′2.4.1 and hydA′2.2 used in the work presented here.

FIGS. 2A-2B show length variation in loop (2A) and stem (2B).

FIG. 3 is an illustration of a model for loop and stem mismatch exclusion. Secondary structure diagrams are shown depicting fingerloop stem-loops binding linear target analogs. A 2D/3D representation of the loop: target complex is also shown.

FIGS. 4A-4B show images contrasting results from experiments at cold incubation (16° C.) (4A) versus room temperature (23° C.) (4B) for the fingerloops (gels at 23° C.) using the fingerloops without tuners (FL DNA hydA′2.2 (FL 7) mismatches −5 to −16). The images demonstrate improved filtering of mismatches. Temperature tunes the stability of the complexes, excluding mismatched targets from probe: target complex formation. P is probe, T target (exact match), C is complex, I is intermediates, NS is nonsense sequence, Mr is molecular weight marker.

FIGS. 5A-5D show images of fingerloop antisense AS18 hydA′2.4.1 (5A) which excludes mismatch and linear antisense (5B) which tolerates mismatch. The mismatch rejection activity of fingerloops is inherent to the structure. AS18 hydA′2.2 (5C) which excludes mismatch and linear antisense (5D) which tolerates mismatch. The mismatch rejection activity of fingerloops is inherent to the structure. P is probe, T target (exact match), C is complex, I is intermediates, NS is nonsense sequence, Mr is molecular weight marker.

FIGS. 6A-6C show gel images of fingerloop FL14 S9 NS9 (6A) which tolerates mismatches and an L18 S9 NS5 beacon-analog probe (6B) that filters out mismatches in a room-temperature annealing experiment with temperature-stabilized gel system (23° C.). (6C) shows gel images of fingerloop FL14 S12 NS8 probe.

FIGS. 7A-7F show images of two beacon analogs of loop 18 (L18) with different size stems wherein both stems are nonsense sequences. (7A) shows probe for loop 18 with nonsense stem sequence 4 bp (L18 NS4). (7B-7C) shows gel images of mismatch tolerance test for L18 NS4. (7D) shows probe for loop 18 nonsense sequence 8 bp (L18 NS8). (7E-7F) shows gel images of mismatch tolerance test for L18 NS8. Only the loop nucleotides are antisense. The images suggest that increasing the length of the nonsense sequence eventually enhances mismatch proofreading during target binding. P is probe, T target (exact match), C is complex, I is intermediates, NS is nonsense sequence, Mr is molecular weight marker.

FIGS. 8A-8B show an illustration of beacon analogs of varying nonsense stem sequences (8A) and gel images of an exact-match-target binding assay for each beacon analog (8B). The images suggest an upper limit of nonsense stem length or stability beyond which beacon-analog probes cannot bind targets. P is probe, T target (exact match), C is complex, I is intermediates, NS is nonsense sequence, Mr is molecular weight marker.

FIGS. 9A-9B show gel images of an L7 probe with a “nonsense” tuner (9A) and without a nonsense tuner (9B). The images suggest removal of the nonsense tuner with matching extension of the antisense sequence prevents the mismatch-filtering activity of the fingerloop. P is probe, P₂ is probe dimer, T target (exact match), CPLX is complex, I is intermediates, NS is nonsense sequence, Mr is molecular weight marker.

FIGS. 10A-10B show (10A) a 7-nt loop fingerloop probe series related by an increasing number of base pairs at the bottom of the stem (“nonsense tuners”). (10B) shows a panel of fingerloop probes with similar free energies that are based on the long AS10 tuner in FIG. 10A. Here, one base pair at a time is converted from nonsense to antisense sequence but with a longer stem basis in the design.

FIGS. 11A-11C show gel images of L7 probes of FIG. 10 with different lengths of “nonsense (NS) tuner” at the base of the helix and their capacity to bind exact-match target while excluding mismatched targets. (11A) the individual probes (NS1-NS10) without annealing partner. (11B) Exact-match target annealing controls (target and probe NS0 alone and together) next to the ladder marker. (11C) The −3 mismatch target binding assay when paired with each member of the probe set with increasing tuners. Gels were run at room temperature. The tuner stabilizes mismatch filtering against temperature change. P is probe, T target (exact match), C is complex, I is intermediates, NS is nonsense sequence, Mr is molecular weight marker.

FIGS. 12A-12J show gel images of the mismatch tolerance test. (12A) individual fingerloops and mutant targets alone and with gel migration controls, then combined with exact-match or point-mutant targets probes NS1 (12B), NS4 (12C), NS7 (12D), NS3 (12E-12F), NS4 (12G-12H), and NS5 (12I-12J). NS3 probe tolerates mismatches, NS4 probe rejects mismatches, permits exact match, blocks point mutant target binding (18nt) NS5 probe rejects exact target, intermediates may include loop-bound target complex. P is probe, P₂ is probe dimer, T target (exact match), C is complex, NS is nonsense sequence, Mr is molecular weight marker.

FIGS. 13A-13D show images demonstrating that stems filter mismatches independently from the loops. (13A) shows an illustration of L7 S15 NS4 fingerloop. (13B) shows an illustration of a “toehold” variant created by incorporating a loop at the base and interrupting the loop at the “top” “circularly permuted probe” CP L7). (13C-13D) show gel images of a mismatch tolerance test for NS 4 circularly-permuted probe. P is probe, T target (exact match), C is complex, NS is nonsense sequence, Mr is molecular weight marker.

FIGS. 14A-14B illustrates the synthesis (14A) and activity (14B) of a fingerloop structure including an antisense sequence region, a nonsense sequence region, and a fluorescent nucleoside at the 3′ end.

FIGS. 15A-15D show the probe set in FIG. 10B (15A) in a gel without annealing partners (purity, migration, dimerization check); (15B) tested against exact match target hydA+, or two different mismatch targets (15C) hydA(−3) and (15D) hydA(−9).

Like reference symbols in the various drawings indicate like elements.

### DETAILED DESCRIPTION

Described herein are detection probes including fingerloop structures described herein and methods of making and using the probes. In some embodiments, described are methods for the detection and amplification of nucleic acids using fingerloop structures described herein. These fingerloop structures can diminish base pairing of a detection probe to a mismatched target nucleic acid. Described are also methods of tuning a fingerloop stability by adding a nonsense sequence after an antisense sequence region. Also disclosed herein are systems and methods to measure the activity of chimeric DNAs in a cell and methods of using these chimeric DNA molecules for measuring and modulating protein expression levels and/or RNA stability from multiple mRNAs in a cell simultaneously.

Reference will now be made in detail to the embodiments of the invention, examples of which are illustrated in the drawings and the examples. This invention may, however, be embodied in many different forms and should not be construed as limited to the embodiments set forth herein.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

A “control” is an alternative subject or sample used in an experiment for comparison purposes. A control can be “positive” or “negative.”

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103.3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. The polynucleotide sequence may be modified, for example, to enhance efficacy and/or to reduce immune responsivity, by using, for example, base modifications or end-capping. In other embodiments, an unmodified polynucleotide sequence is used. For example, the polynucleotide can be an RNA sequence or a DNA sequence. In some embodiments, the mRNA can include an optimized codon. By codon optimizing, the formation of secondary structures can be reduced and translational efficiency improved. In certain embodiments, the codon optimization includes GC enrichment of the coding region. In certain embodiments, the codon optimization includes codon quality enrichment of the coding region. In certain aspects, the mRNA can include one or more regions or parts, which act or function as an untranslated region (UTRs) of a gene. UTRs are transcribed but not translated. In mRNA, the S′ UTR starts at the transcription start site and continues to the start codon but does not include the start codon. The 3′ UTR starts immediately following the stop codon and continues until the transcriptional termination signal. The use of human-derived UTRs may facilitate the expression of the polypeptide in cells. In some embodiments, the polynucleotide comprises at least one chemically modified nucleotide. In some embodiments, the at least one chemically modified nucleotide comprises a chemically modified nucleobase, a chemically modified ribose, a chemically modified phosphodiester linkage, or a combination thereof. In some embodiments, the polynucleotide sequence as used comprise modified nucleosides such as 5-methylcystonsine or pseudouridine.

As used herein “modified” refers to a changed state or structure of a molecule of the invention. Molecules may be modified in many ways including chemically, structurally, and functionally. In one embodiment, the polynucleotides of the present invention are “chemically modified” by the introduction of non-natural nucleosides and/or nucleotides, e.g., as it relates to the natural ribonucleotides A, U, G, and C. Modifications of the nucleosides and/or nucleotides as used in the present invention may be naturally occurring (i.e. comprise a nucleotide and/or nucleoside other than the natural ribonucleotides A, U, G, and C) or may be artificial. Non-canonical nucleotides such as the cap structures are not considered “modified” although they differ from the chemical structure of A, G, C, and U ribonucleotides. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. When the polynucleotides of the present invention are chemically and/or structurally modified, the polynucleotides may be referred to as “modified nucleotides”.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The term “polypeptide” refers to a compound made up of a single chain of D- or L-amino acids or a mixture of D- and L-amino acids joined by peptide bonds.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures or a chemical denaturant such as urea, guanidinium, or dimethyl sulfoxide, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), translation initiation regions (TIRs), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g, tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-US' segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA, Vol. 78 (3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e, about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequent coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990). J. Mol. Biol. 215.403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength of 3, an expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in the same reading frame. However, operably linked nucleic acids (e.g. enhancers and coding sequences) do not have to be contiguous. Linking can be accomplished by ligation at convenient restriction sites, Gibson synthesis, or CRISPR editing. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter). In some embodiments, operably linked nucleic acids can include chimeric nucleic acids (wherein the linked nucleic acid sequences are not naturally fused or linked together).

The term “gene leader sequence” refers to the portion of a gene that encodes for an mRNA leader sequence. The term “mRNA leader sequence” refers to the portion of an mRNA sequence that is upstream from the start of the protein coding sequence portion of the mRNA. The gene leader sequence includes, for example, the translation initiation region (TIR).

As used herein, the term “fingerloop” refers to a structure formed by an intramolecular base pairing when a nucleotide sequence and a complementary sequence thereof is present in reverse direction in the same strand and a non-complementary sequence is present there between in the same strand. The fingerloop structure comprises an antisense sequence region that binds to the target nucleic acid sequence, and a nonsense sequence region, where the antisense region is located in the loop and in one strand of the stem loop of the fingerloop structure. For example, fingerloops can refer to the stem-loop antisense motifs of engineered DsrA that provide a modular, general purpose RNA antisense-encoding structure. For example, these fingerloops can also use DNA in place of RNA. The length of the fingerloop nucleotide sequence may be, for example, in the range from 10 nt to 150 nt, 10 nt to 100 nt, 10 nt to 75 nt, 10 nt to 50 nt, or 10 nt to 25 nt.

As used herein, the term “sRNA” or “small regulatory RNA” refers to a short-length RNA, which is usually 300 or less nucleotides in length, is not generally translated into protein, and effectively inhibits the translation and/or stability of a specific mRNA by complementary binding.

The term “mismatched” or “mismatched target sequence” refers to an off-target sequence that is not perfectly complementary to the first DNA sequence or the second DNA sequence of the chimeric deoxyribonucleic acid described herein. The dual retargeted DNA may have at least one mismatch, but can also have 2, 3, 4, 5, 6 or 7 or more mismatched nucleotides to the off-target sequence.

As used herein, the term “molecular beacon” refers a to detectable molecule, where the detectable property of the molecule is detectable only under certain specific conditions, thereby enabling it to function as a specific and informative signal Non-limiting examples of detectable properties are, optical properties, electrical properties, magnetic properties, chemical properties and time or speed through an opening of known size. In some embodiments a molecular beacon can be a single-stranded oligonucleotide capable of forming a stem-loop structure, where the loop sequence may be complementary to a target nucleic acid sequence of interest and is Banked by short complementary arms that can form a stem. The oligonucleotide may be labeled at one end with a fluorophore and at the other end with a quencher molecule. In the stem-loop conformation, energy from the excited fluorophore is transferred to the quencher, through long-range dipole-dipole coupling similar to that seen in fluorescence resonance energy transfer, or FRET, and released as heat instead of light. When the loop sequence is hybridized to a specific target sequence, the two ends of the molecule are separated and the energy from the excited fluorophore is emitted as light, generating a detectable signal.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Reference will now be made in detail to specific aspects of the disclosed materials, compounds, compositions, articles, and methods, examples of which are illustrated in the accompanying Examples and Figures.

### Fingerloop Structure

Described herein are fingerloop structures including: an antisense sequence region located in a loop and in one strand of a stem loop of the fingerloop structure, and a nonsense sequence region located at the base of the stem loop of the fingerloop structure. In some embodiments, described herein are fingerloop structures including: an antisense sequence region located in a loop and in one strand of a stem loop of the fingerloop structure; and a nonsense sequence region located at the base of the stem loop of the fingerloop structure. In some embodiments, the antisense sequence region can be complementary to a target nucleic acid sequence. In some embodiments, the one strand of the stem loop can be a descending strand of the fingerloop structure. In some embodiments, the one strand of the stem loop can be an ascending strand of the fingerloop structure.

In some embodiments, when the one strand of the stem loop is a descending strand of the fingerloop structure, then the nonsense sequence can be located at a 3′ end of the descending strand. In some embodiments, when the one strand of the stem loop is an ascending strand of the fingerloop structure, then the nonsense sequence can be located at a S′ end of the ascending strand.

In some embodiments, when the one strand of the stem loop is a descending strand of the fingerloop structure, then the nonsense sequence can be located before the antisense sequence region at a 3′ end of the descending strand. In some embodiments, when the one strand of the stem loop is an ascending strand of the fingerloop structure, then the nonsense sequence can be located after the antisense sequence region at a 5′ end of the ascending strand.

In some embodiments, the nucleic acid sequence can further include a fluorescent nucleoside at a 3′ end of the ascending strand of the fingerloop structure. In some embodiments, the nucleic acid sequence can further include a fluorescent nucleoside at a 5′ end of the descending strand of the fingerloop structure. Suitable fluorescent nucleosides are described in U.S. Patent Publication No. 2008/0261823 and Sholokh et al. 2015, JACS 137:3185-3188. In some embodiments, the fluorescent nucleoside can be thienoguanosine, thienouricil, 2-aminopurine, or 6-methyl isoxanthopterin (6-MI).

In some embodiments, the nucleic acid sequence can further include nucleotides labeled at one end with a fluorophore and at the other end with a quencher molecule (e.g., DABCYL) In the stem-loop conformation, energy from the excited fluorophore is transferred to the quencher, through long-range dipole-dipole coupling similar to that seen in fluorescence resonance energy transfer, or FRET, and released as heat instead of light. When the antisense sequence is hybridized to a specific target sequence, the two ends of the molecule are separated and the energy from the excited fluorophore is emitted as light, generating a detectable signal.

In some embodiments, the fingerloop structure diminishes base pairing to a mismatched target nucleic acid. In some embodiments, the fingerloop structure provides increased detection specificity of the target nucleic acid sequence. In some embodiments, fingerloop structure improves targeting fidelity. In some embodiments, fingerloop structure decrease false positive results.

In some embodiments, the antisense sequence region can include at least 9 nucleotides, (e.g., at least 10 nucleotides, at least 15 nucleotides, at least 20 nucleotides, at least 25 nucleotides, at least 30 nucleotides, or at least 35 nucleotides). In some embodiments, the antisense sequence region can include 40 nucleotides or less, (e.g., 35 nucleotides or less, 30 nucleotides or less, 25 nucleotides or less, 20 nucleotides or less, 15 nucleotides or less, or 10 nucleotides or less).

The antisense sequence region can range from any of the minimum values described above to any of the maximum values described above. For example, in some embodiments, the antisense sequence region can include from 9 to 40 nucleotides, (e.g., from 10 to 40 nucleotides, from 15 to 40 nucleotides, from 20 to 40 nucleotides, from 25 to 40 nucleotides, from 30 to 40 nucleotides, or from 35 to 40 nucleotides).

In some embodiments, the antisense sequence region of the loop can include at least 3 nucleotides (e g., at least 4 nucleotides, at least S nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, or at least 19 nucleotides). In some embodiments, the antisense sequence region of the loop can include 20 nucleotides or less, (e.g., 19 nucleotides or less, 18 nucleotides or less, 17 nucleotides or less, 16 nucleotides or less, 15 nucleotides or less, 14 nucleotides or less, 13 nucleotides or less, 12 nucleotides or less, 11 nucleotides or less, 10 nucleotides or less, 9 nucleotides or less, 8 nucleotides or less, 7 nucleotides or less, 6 nucleotides or less, 5 nucleotides or less, or 4 nucleotides or less).

The antisense sequence region of the loop ranging from any of the minimum values described above to any of the maximum values described above. For example, in some embodiments, the antisense sequence region of the loop can include from 3 to 20 nucleotides, (e.g., from 5 to 20 nucleotides, from 10 to 20 nucleotides, from 15 to 20 nucleotides, from 5 to 15 nucleotides, from 5 to 10 nucleotides, or from 10 to 15 nucleotides).

In some embodiments, the antisense sequence region of the stem loop can include at least 6 nucleotides in one strand of a stem loop, (e.g., at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, or at least 19 nucleotides). In some embodiments, the antisense sequence region of the stem loop can include 20 nucleotides or less, (e.g., 19 nucleotides or less, 18 nucleotides or less, 17 nucleotides or less, 16 nucleotides or less, 15 nucleotides or less, 14 nucleotides or less, 13 nucleotides or less, 12 nucleotides or less, 11 nucleotides or less, 10 nucleotides or less, 9 nucleotides or less, 8 nucleotides or less, or 7 nucleotides or less).

The antisense sequence region of the stem loop ranging from any of the minimum values described above to any of the maximum values described above. For example, in some embodiments, the antisense sequence region of the stem loop can include from 6 to 20 nucleotides in one strand of a stem loop, (e.g., from 10 to 20 nucleotides, from 15 to 20 nucleotides, from 6 to 10 nucleotides, or from 10 to 15 nucleotides, or from 6 to 15 nucleotides).

In some embodiments, the nonsense sequence region of the stem loop can include at least 1 nucleotide in one strand of a stem loop (e.g., at least 2 nucleotides, at least 3 nucleotides, at least 4 nucleotides, at least 5 nucleotides, at least 6 nucleotides, at least 7 nucleotides, at least 8 nucleotides, or at least 9 nucleotides). In some embodiments, the nonsense sequence region of the stem loop can include 10 nucleotides or less in one strand of a stem loop (e.g., 9 nucleotides or less, 8 nucleotides or less, 7 nucleotides or less, 6 nucleotides or less, 5 nucleotides or less, 4 nucleotides or less, 3 nucleotides or less, or 2 nucleotides or less).

The nonsense sequence region of the stem loop ranging from any of the minimum values described above to any of the maximum values described above. For example, in some embodiments, the nonsense sequence region of the stem loop can include from 1 to 10 nucleotides in one strand of a stem loop (e.g., from 2 to 10 nucleotides, from 2 to 5 nucleotides, from 3 to 10 nucleotides, from 3 to 5 nucleotides, from 4 to 10 nucleotides, from 4 to 5 nucleotides, from 5 to 10 nucleotides, from 5 to 8 nucleotides, from 6 to 10 nucleotides, from 6 to 8 nucleotides, from 7 to 10 nucleotides, from 8 to 10 nucleotides, or from 9 to 10 nucleotides).

In one aspect, it is contemplated herein that the length of the loops, stem loop of the fingerloop can be adjusted to optimize the scaffold. For example, the scaffold can be shortened, not adjusted, or extended at the loop by 1, 2, 3, 4, 5, 6, 7, 8, 9, 10, 11, 12, 13, 14, 15, 16, 17, 18, 19, or 20 nucleotides. Thus, in one aspect, the loop can be 5, 6, 7, 8, 9, 10, 11, 12, 13, 14, 15, 16, 17, 18, 19, 20, 21, 22, 23, 24, 25 nucleotides long. Similarly, the stem can be shortened, not adjusted, or extended by 1, 2, 3, 4, 5, 6, 7, 8, 9, 10, 11, 12, 13, 14, 15, 16, 17, 18, 19, or 20 nucleotides long.

In some embodiments, the fingerloop structure can have a ΔG of at least −2 kcal/mol, (e.g., at least −2 kcal/mol, at least −3 kcal/mol, at least −4 kcal/mol, at least −5 kcal/mol, at least −6 kcal/mol, at least −7 kcal/mol, at least −8 kcal/mol, at least −9 kcal/mol, at least −10 kcal/mol, at least −11 kcal/mol, at least −12 kcal/mol, at least −13 kcal/mol, at least −14 kcal/mol, at least −15 kcal/mol, at least −16 kcal/mol, at least −17 kcal/mol, at least −18 kcal/mol, at least −19 kcal/mol, at least −20 kcal/mol, at least −21 kcal/mol, at least −22 kcal/mol, at least −23 kcal/mol, at least −24 kcal/mol, at least −25 kcal/mol, at least −26 kcal/mol, at least −27 kcal/mol, at least −28 kcal/mol, or at least −29 kcal/mol).

In some embodiments, the fingerloop structure can have a ΔG of −30 kcal/mol or less, (e.g., −29 kcal/mol or less, −28 kcal/mol or less, −27 kcal/mol or less, −26 kcal/mol or less, −25 kcal/mol or less, −24 kcal/mol or less, −23 kcal/mol or less, −22 kcal/mol or less, −21 kcal/mol or less, −20 kcal/mol or less, −19 kcal/mol or less, −19 kcal/mol or less, −19 kcal/mol or less, −18 kcal/mol or less, −17 kcal/mol or less, −16 kcal/mol or less, −15 kcal/mol or less, −14 kcal/mol or less, −13 kcal/mol or less, −12 kcal/mol or less, −11 kcal/mol or less, −10 kcal/mol or less, −9 kcal/mol or less, −8 kcal/mol or less, −7 kcal/mol or less, −6 kcal/mol or less, −5 kcal/mol or less, −4 kcal/mol or less, or −3 kcal/mol or less).

The fingerloop structure can have a ΔG ranging from any of the minimum values described above to any of the maximum values described above. For example, in some embodiments, the fingerloop structure can have a ΔG of from −2 kcal/mol to −30 kcal/mol, (e.g., from −2 kcal/mol to −20 kcal/mol, from −5 kcal/mol to −18 kcal/mol, from −5 kcal/mol to −20 kcal/mol, from −5 kcal/mol to −30 kcal/mol, from −8 kcal/mol to −15 kcal/mol, from −8 kcal/mol to −18 kcal/mol, from −8 kcal/mol to −30 kcal/mol, from −10 kcal/mol to −30 kcal/mol, from −10 kcal/mol to −20 kcal/mol, from −10 kcal/mol to −15 kcal/mol, from −10 kcal/mol to −18 kcal/mol, from −15 kcal/mol to −18 kcal/mol, from −15 kcal/mol to −20 kcal/mol, from −15 kcal/mol to −30 kcal/mol, from −20 kcal/mol to −30 kcal/mol, or from −25 kcal/mol to −30 kcal/mol).

In some embodiments, the nucleic acid can include RNA, DNA, RNA derivatives, DNA derivatives, or any combination thereof. For example, suitable nucleic acids can include but are not limited to LNAs, PNAs, 2′-Fluoro RNAs, or other commonly used synthetic nucleic acid analogs.

In some embodiments, the nucleic acid sequences can include modified nucleotides. Various chemically modified nucleotides are known in the art, for example, see WO/2018/009822.

In one embodiment, the at least one chemically modified nucleotide is a chemically modified ribose. In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O)-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is selected from 2′-O-methyl (2′-O-Me) or 2′-Fluoro (2′-F). In one embodiment, the chemically modified ribose is 2′-O-methyl (2′-O-Me). In one embodiment, the chemically modified ribose is 2′-Fluoro (2′-F).

In one embodiment, the at least one chemically modified nucleotide is a chemically modified nucleobase. In one embodiment, the chemically modified nucleobase is selected from S-formylcytidine (5fC), 5-methylcytidine (5meC), 5-methoxycytidine (5moC), 5-hydroxycytidine (5hoC), S-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In one embodiment, the at least one chemically modified nucleotide is a chemically modified phosphodiester linkage. In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), 2′,5′-phosphodiester (2′,5′-PO) or morpholino (phosphorodiamidate morpholino oligomer). In one embodiment, the chemically modified phosphodiester linkage is phosphorothioate.

Other modified nucleoside analogues can be found in U.S. Patent Publication No. 2008/0261823.

### Detection Probes

Described herein are detection probes including: a nucleic acid sequence including a fingerloop structure described herein. In some embodiments, the detection probe diminishes base pairing to a mismatched target nucleic acid.

In some embodiments, the detection probe can be a Northern blot probe, a Southern blot probe, or any combination thereof. In some embodiments, the detection probe can be a Northern blot probe. In some embodiments, the detection probe can be a Southern blot probe.

In some embodiments, the detection probe can have a ΔG of at least −2 kcal/mol, (e.g., at least −2 kcal/mol, at least −3 kcal/mol, at least −4 kcal/mol, at least −5 kcal/mol, at least −6 kcal/mol, at least −7 kcal/mol, at least −8 kcal/mol, at least −9 kcal/mol, at least −10 kcal/mol, at least −11 kcal/mol, at least −12 kcal/mol, at least −13 kcal/mol, at least −14 kcal/mol, at least −15 kcal/mol, at least −16 kcal/mol, at least −17 kcal/mol, at least −18 kcal/mol, at least −19 kcal/mol, at least −20 kcal/mol, at least −21 kcal/mol, at least −22 kcal/mol, at least −23 kcal/mol, at least −24 kcal/mol, at least −25 kcal/mol, at least −26 kcal/mol, at least −27 kcal/mol, at least −28 kcal/mol, or at least −29 kcal/mol).

In some embodiments, the detection probe can have a ΔG of −30 kcal/mol or less, (e.g., −29 kcal/mol or less, −28 kcal/mol or less, −27 kcal/mol or less, −26 kcal/mol or less, −25 kcal/mol or less, −24 kcal/mol or less, −23 kcal/mol or less, −22 kcal/mol or less, −21 kcal/mol or less, −20 kcal/mol or less, −19 kcal/mol or less, −19 kcal/mol or less, −19 kcal/mol or less, −18 kcal/mol or less, −17 kcal/mol or less, −16 kcal/mol or less, −15 kcal/mol or less, −14 kcal/mol or less, −13 kcal/mol or less, −12 kcal/mol or less, −11 kcal/mol or less, −10 kcal/mol or less, −9 kcal/mol or less, −8 kcal/mol or less, −7 kcal/mol or less, −6 kcal/mol or less, −5 kcal/mol or less, −4 kcal/mol or less, or −3 kcal/mol or less).

The detection probe can have a ΔG ranging from any of the minimum values described above to any of the maximum values described above. For example, in some embodiments, the detection probe can have a ΔG of from −2 kcal/mol to −30 kcal/mol, (e.g., from −2 kcal/mol to −20 kcal/mol, from −5 kcal/mol to −18 kcal/mol, from −5 kcal/mol to −20 kcal/mol, from −5 kcal/mol to −30 kcal/mol, from −8 kcal/mol to −15 kcal/mol, from −8 kcal/mol to −18 kcal/mol, from −8 kcal/mol to −30 kcal/mol, from −10 kcal/mol to −30 kcal/mol, from −10 kcal/mol to −20 kcal/mol, from −10 kcal/mol to −15 kcal/mol, from −10 kcal/mol to −18 kcal/mol, from −15 kcal/mol to −18 kcal/mol, from −15 kcal/mol to −20 kcal/mol, from −15 kcal/mol to −30 kcal/mol, from −20 kcal/mol to −30 kcal/mol, or from −25 kcal/mol to −30 kcal/mol).

### Compositions

Described herein are also composition including a detection probe described herein herein and a pharmaceutically acceptable carrier. Also described herein are also composition including a fingerloop structure described herein and a pharmaceutically acceptable carrier.

The term “pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol busks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include etbylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, tale, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

In some embodiments, suitable detection probes delivery vehicles can include, but are not limited to lipid-based (e.g., a liposome formulation, lipoplexes, or lipid nanoparticles (LNP)), viral-based, or physical methods such as injection, microinjection, electroporation, ultrasound, gene gun, hydrodynamic applications, or any combination thereof.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables.

In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

The compounds (e.g., detection probes) can be incorporated microparticles, nanoparticles, or combinations thereof that provide controlled release of the compounds and/or additional active agents. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the detection probes can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of detection probes into carrier materials to produce detection probes-containing microparticles can be achieved through known pharmaceutical formulation techniques. In the case of formulation in fats, waxes or wax-like materials, the carrier material is typically heated above its melting temperature and the drug is added to form a mixture comprising particles suspended in the carrier material, detection probes dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce detection probes-containing microparticles. In this case detection probes and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, detection probes in a particulate form is homogeneously dispersed in a water-insoluble or slowly water soluble material. To minimize the size of the particles within the composition, the powder itself may be milled to generate fine particles prior to formulation. The process of jet milling, known in the pharmaceutical art, can be used for this purpose. In some embodiments, detection probes in a particulate form is homogeneously dispersed in a wax or wax like substance by heating the wax or wax like substance above its melting point and adding the particles while stirring the mixture. In this case a pharmaceutically acceptable surfactant may be added to the mixture to facilitate the dispersion of the particles. The particles can also be coated with one or more modified release coatings. Solid esters of fatty acids, which are hydrolyzed by lipases, can be spray coated onto microparticles or particles. Zein is an example of a naturally water-insoluble protein. It can be coated onto drug containing microparticles or particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding detection probes containing microparticles or particles, a water-soluble protein can be spray coated onto the microparticles and subsequently cross-linked by the one of the methods described above. Alternatively, detection probes-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

The detection probes described herein can be incorporated into injectable/implantable solid or semi-solid implants, such as polymeric implants. In one embodiment, the detection probes are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the detection probes dissolved or dispersed in the polymer solution and the solvent is then evaporated. Solvent processes require that the polymer be soluble in organic solvents. Another method is compression molding of a mixed powder of the polymer and the polymer particles loaded with the detection probes.

Alternatively, the detection probes can be incorporated into a polymer matrix and molded, compressed, or extruded into a device that is a solid at room temperature. For example, the detection probes can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the detection probes from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the detection probes from the implant are well known in the art.

In some embodiments, the detection probes are incorporated in a delivery system such as gels, nanoparticles, microparticles, or implants such as (e.g, rods, discs, wafers, orthopedic implants) for sustained release. In some embodiments, the detection probes can be administered using a local delivery implantable system comprising the detection probes incorporated within a gel, nanoparticles, microparticles, or an implant. In some embodiments, the compositions comprise a delivery system such as gels, nanoparticles, microparticles, or implants such as (e.g., rods, discs, wafers, orthopedic implants) for sustained release of the detection probes.

In some embodiments, the composition can be a nanoparticle.

### Methods

Described herein are also methods for detecting a target nucleic acid sequence, the method including: providing a nucleic acid sequence; contacting the nucleic acid sequence with a detection probe described herein; assessing a change in fluorescence intensity. In some embodiments, an increase in fluorescence intensity indicates binding of the detection probe to a target nucleic acid sequence.

Described herein are also methods of tuning a fingerloop stability, the method including: adding a nonsense nucleic acid sequence to a nucleic acid sequence including a fingerloop structure including: an antisense sequence region located in a loop and in one strand of a stem loop of the fingerloop structure. In some embodiments, the antisense sequence region is complementary to a target nucleic acid sequence. In some embodiments, the nonsense nucleic acid sequence can be located at the base of the stem loop of the fingerloop structure. In some embodiments, the one strand of the stem loop can be a descending strand of the fingerloop structure. In some embodiments, the one strand of the stem loop can be an ascending strand of the fingerloop structure.

In some embodiments, when the one strand of the stem loop is a descending strand of the fingerloop structure, then the nonsense sequence can be located at a 3′ end of the descending strand. In some embodiments, when the one strand of the stem loop is an ascending strand of the fingerloop structure, then the nonsense sequence can be located at a 5′ end of the ascending strand.

In some embodiments, when the one strand of the stem loop is a descending strand of the fingerloop structure, then the nonsense sequence can be located before the antisense sequence region at a 3′ end of the descending strand. In some embodiments, when the one strand of the stem loop is an ascending strand of the fingerloop structure, then the nonsense sequence can be located after the antisense sequence region at a 5′ end of the ascending strand.

In some embodiments, the fingerloop structure stability can be maintained despite temperature changes. In some embodiments, tuning fingerloop stability improves targeting fidelity. In some embodiments, tuning fingerloop stability decrease false positive results. In some embodiments, the method can tune fingerloop activity by tuning fingerloop stability.

In some embodiments, the antisense sequence region can include at least 9 nucleotides, (e.g., at least 10 nucleotides, at least 15 nucleotides, at least 20 nucleotides, at least 25 nucleotides, at least 30 nucleotides, or at least 35 nucleotides). In some embodiments, the antisense sequence region can include 40 nucleotides or less, (e.g., 35 nucleotides or less, 30 nucleotides or less, 25 nucleotides or less, 20 nucleotides or less, 15 nucleotides or less, or 10 nucleotides or less).

The antisense sequence region ranging from any of the minimum values described above to any of the maximum values described above. For example, in some embodiments, the antisense sequence region can include from 9 to 40 nucleotides, (e.g., from 10 to 40 nucleotides, from 15 to 40 nucleotides, from 20 to 40 nucleotides, from 25 to 40 nucleotides, from 30 to 40 nucleotides, or from 35 to 40 nucleotides).

In some embodiments, the antisense sequence region of the loop can include at least 3 nucleotides (e.g., at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, or at least 19 nucleotides). In some embodiments, the antisense sequence region of the loop can include 20 nucleotides or less, (e.g., 19 nucleotides or less, 18 nucleotides or less, 17 nucleotides or less, 16 nucleotides or less, 15 nucleotides or less, 14 nucleotides or less, 13 nucleotides or less, 12 nucleotides or less, 11 nucleotides or less, 10 nucleotides or less, 9 nucleotides or less, 8 nucleotides or less, 7 nucleotides or less, 6 nucleotides or less, 5 nucleotides or less, or 4 nucleotides or less).

The antisense sequence region of the loop ranging from any of the minimum values described above to any of the maximum values described above. For example, in some embodiments, the antisense sequence region of the loop can include from 3 to 20 nucleotides, (e.g., from 5 to 20 nucleotides, from 10 to 20 nucleotides, from 15 to 20 nucleotides, from 5 to 15 nucleotides, from 5 to 10 nucleotides, or from 10 to 15 nucleotides).

In some embodiments, the antisense sequence region of the stem loop can include at least 6 nucleotides in one strand of a stem loop, (e.g., at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, or at least 19 nucleotides). In some embodiments, the antisense sequence region of the stem loop can include 20 nucleotides or less in one strand of a stem loop, (e.g., 19 nucleotides or less, 18 nucleotides or less, 17 nucleotides or less, 16 nucleotides or less, 15 nucleotides or less, 14 nucleotides or less, 13 nucleotides or less, 12 nucleotides or less, 11 nucleotides or less, 10 nucleotides or less, 9 nucleotides or less, 8 nucleotides or less, or 7 nucleotides or less).

The antisense sequence region of the stem loop ranging from any of the minimum values described above to any of the maximum values described above. For example, in some embodiments, the antisense sequence region of the stem loop can include from 6 to 20 nucleotides in one strand of a stem loop, (e.g., from 10 to 20 nucleotides, from 15 to 20 nucleotides, from 6 to 10 nucleotides, or from 10 to 15 nucleotides, or from 6 to 15 nucleotides).

In some embodiments, the nonsense sequence region of the stem loop can include at least 1 nucleotide in one strand of a stem loop (e.g., at least 2 nucleotides, at least 3 nucleotides, at least 4 nucleotides, at least 5 nucleotides, at least 6 nucleotides, at least 7 nucleotides, at least 8 nucleotides, or at least 9 nucleotides). In some embodiments, the nonsense sequence region of the stem loop can include 10 nucleotides or less in one strand of a stem loop (e.g., 9 nucleotides or less, 8 nucleotides or less, 7 nucleotides or less, 6 nucleotides or less, 5 nucleotides or less, 4 nucleotides or less, 3 nucleotides or less, or 2 nucleotides or less).

The nonsense sequence region of the stem loop ranging from any of the minimum values described above to any of the maximum values described above. For example, in some embodiments, the nonsense sequence region of the stem loop can include from 1 to 10 nucleotides in one strand of a stem loop (e.g., from 2 to 10 nucleotides, from 2 to 5 nucleotides, from 3 to 10 nucleotides, from 3 to 5 nucleotides, from 4 to 10 nucleotides, from 4 to 5 nucleotides, from 5 to 10 nucleotides, from 5 to 8 nucleotides, from 6 to 10 nucleotides, from 6 to 8 nucleotides, from 7 to 10 nucleotides, from 8 to 10 nucleotides, or from 9 to 10 nucleotides).

Described herein are also methods for amplifying a nucleic acid sequence including: providing a nucleic acid sample; amplifying a target nucleic acid sequence from the nucleic acid sample using amplification primers in a polymerase chain reaction, wherein the amplification primers includes a fingerloop structure described herein. In some embodiments, described herein the methods for amplifying a nucleic acid sequence include: providing a nucleic acid sample; amplifying a target nucleic acid sequence from the nucleic acid sample using amplification primers in a polymerase chain reaction, wherein the amplification primers includes a fingerloop structure including: an antisense sequence region located in a loop and in one strand of a stem loop of the fingerloop structure; and a nonsense sequence region located at the base of the stem loop of the fingerloop structure. In some embodiments, the antisense sequence region can be complementary to the target nucleic acid sequence. In some embodiments, the fingerloop structure diminishes base pairing to a mismatched target nucleic acid.

In some embodiments, the amplification primers can include at least 25 nucleotides, (e.g., at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, or at least 95 nucleotides).

In some embodiments, the amplification primers can include 100 nucleotides or less, (e.g., 95 nucleotides or less, 90 nucleotides or less, 85 nucleotides or less, 80 nucleotides or less, 75 nucleotides or less, 70 nucleotides or less, 65 nucleotides or less, 60 nucleotides or less, 55 nucleotides or less, 50 nucleotides or less, 45 nucleotides or less, 40 nucleotides or less, 35 nucleotides or less, 30 nucleotides or less, or 25 nucleotides or less).

The amplification primers ranging from any of the minimum values described above to any of the maximum values described above. For example, in some embodiments, the amplification primers can include from about 25 to about 100 nucleotides.

In some embodiments, the amplification is a standard polymerase chain reaction (PCR) reaction. In some embodiments, the amplification is a real-time PCR reaction. In some embodiments, the amplification is a reverse-transcription PCR reaction. In some embodiments, the amplification is a quantitative reverse-transcription PCR (qRT-PCR) reaction.

The improved nucleic acid detection methods disclosed herein can be used in a number of detection technologies. In some embodiments, the fingerloops can be used to detect microorganism or pathogens in a sample. In some embodiments, the fingerloops can be used to detect a DNA sequence. In some embodiments, the fingerloops are used in detection probes for Southern blots. In some embodiments, the fingerloops can be used to detect an RNA sequence. In some embodiments, the fingerloops are used in detection probes for Northern blots. In some embodiments, the fingerloops are used in detection probes for detection of mRNAs. In yet other embodiments, the fingerloop structures are used as therapeutics, such as cancer drug delivery and mRNA-triggered nanoparticle function, as well as species-specific identification of (e.g. gut microbiome bacteria), antivirals, for example, by binding to key sequences of an RNA virus or DNA virus to inhibit packaging or some other essential function for viral replication.

In some aspects, disclosed herein is a system or a kit for measuring the activity of a chimeric deoxyribonucleic acid (DNA) in a cell, the system including a chimeric DNA, wherein the chimeric DNA including a first deoxyribonucleic acid (DNA) sequence operably linked to a second deoxyribonucleic acid (DNA) sequence; a first plasmid including a first reporter gene operably linked to a first gene leader sequence; and a second plasmid including a second reporter gene operably linked to a second gene leader sequence. In some embodiments, the first DNA sequence can be present in a first fingerloop structure described herein and the second DNA sequence can be present in a second fingerloop structure described herein. In some embodiments, the first fingerloop structure can include: an antisense sequence region located in a loop and in one strand of a stem loop of the fingerloop structure; and a nonsense sequence region located at the base of the stem loop of the fingerloop structure. In some embodiments, the second fingerloop structure can include: an antisense sequence region located in a loop and in one strand of a stem loop of the fingerloop structure, and a nonsense sequence region located after at the base of the stem loop of the fingerloop structure. In some embodiments, the first and second fingerloop inhibits the binding of the first and second DNAs to mismatched target sequences.

Also, described herein are methods for modulating protein expression levels and/or mRNA expression levels from at least two target mRNAs in a cell simultaneously, the method including: transforming the cell with a system for measuring the activity of a chimeric deoxyribonucleic acid (DNA), and measuring the protein expression levels and/or mRNA expression levels of the first reporter gene and the second reporter gene.

In some embodiments, the system can include: a chimeric DNA, wherein the chimeric DNA including a first deoxyribonucleic acid (DNA) sequence operably linked to a second deoxyribonucleic acid (DNA) sequence; a first plasmid including a first reporter gene operably linked to a first gene leader sequence; and a second plasmid including a second reporter gene operably linked to a second gene leader sequence. In some embodiments, the first DNA sequence can be present in a first fingerloop structure described herein and the second DNA sequence can be present in a second fingerloop structure described herein. In some embodiments, the first fingerloop structure can include: an antisense sequence region located in a loop and in one strand of a stem loop of the fingerloop structure; and a nonsense sequence region located at the base of the stem loop of the fingerloop structure. In some embodiments, the antisense sequence region is complementary to an mRNA of the first gene leader sequence. In some embodiments, the second fingerloop structure can include: an antisense sequence region located in a loop and in one strand of a stem loop of the fingerloop structure, and a nonsense sequence region located at the base of the stem loop of the fingerloop structure. In some embodiments, the antisense sequence region can be complementary to an mRNA of the second gene leader sequence.

In some embodiments, the first and second fingerloop inhibits the binding of the first and second DNAs to mismatched target sequences.

In some embodiments, the chimeric DNA is from about 50 to about 300 nucleotides in length. In some embodiments, the chimeric DNA is from about 50 to about 100, from about 50 to about 150, from about 50 to about 200, from about 50 to about 250, or from about 50 to about 300, nucleotides in length.

In some embodiments, the cell is an Escherichia coli (E. coli) cell. In some embodiments, the cell is a Bacillus subtilis (B. subtilis) cell. In some embodiments, the cell is a Clostridium acetobutylicum (C. acetobutylicum) cell. In some embodiments, the cell can be any suitable prokaryotic cell. In some embodiments, the chimeric (fingerloop) DNAs are used to test exogenous sequences in E. coli.

In some embodiments, the chimeric DNA binds to the at least two target mRNAs encoding at least two cell enzymes, and wherein binding results in a reduction of activity of the at least two cell enzymes.

In some embodiments, the at least two target mRNAs are in the same metabolic pathway. In some embodiments, the at least two target mRNAs are in different metabolic pathways.

In some embodiments, the chimeric DNA comprises a fingerloop structure described herein. In some embodiments, the first DNA sequence and the second DNA sequence are comprised in stem-loop antisense structures. In some embodiments, the first DNA sequence and the second DNA sequence are comprised in at least two fingerloop structures.

In some embodiments, the first DNA sequence is present in a descending strand of the first fingerloop structure. In some embodiments, the first DNA sequence is present in an ascending strand of the first fingerloop structure. In some embodiments, the second DNA sequence is present in a descending strand of the second fingerloop structure. In some embodiments, the second DNA sequence is present in an ascending strand of the second fingerloop structure. In some embodiments, the first and second DNA sequences are positioned in antisense sequence region of the fingerloop structure.

In some embodiments, the first DNA sequence binds to an mRNA of the first gene leader sequence. In some embodiments, the second DNA sequence binds to an mRNA of the second gene leader sequence.

In some embodiments, the first reporter gene encodes a fluorescent protein. In some embodiments, the second reporter gene encodes a fluorescent protein. In some embodiments, the reporter gene is a non-fluorescent protein.

In some embodiments, the first reporter gene encodes a GFP protein. In some embodiments, the first reporter gene encodes an mCherry protein. In some embodiments, the second reporter gene encodes a GFP protein. In some embodiments, the second reporter gene encodes an mCherry protein.

In some embodiments, the first and second gene leader sequences target genes in the same metabolic pathway. In some embodiments, the first and second gene leader sequences target genes in different metabolic pathways. For example, the systems herein can be used to alter ATP levels while improving yield of a specific metabolite in a different pathway.

In some embodiments, the chimeric DNA binds to the at least two target mRNAs encoding at least two endogenous cell enzymes, and wherein binding results in a reduction of activity in the cell of the at least two cell enzymes. In some embodiments, the reduction in activity occurs due to the decrease in translation (and does not affect the enzyme's rate of activity directly). In some embodiments, the chimeric DNA binds to the at least two target mRNAs encoding at least two heterologous cell enzymes. In some embodiments, the chimeric DNA binds to the at least two target mRNAs encoding at least two endogenous cell enzymes.

In some embodiments, the chimeric DNA affects mRNA expression levels by modulating the stability of the target mRNA. In some embodiments, the chimeric DNA affects mRNA expression levels by blocking the access of the ribosome.

One of the important features of the fingerloop structure is that it acts as a modular unit of antisense sequence that can be targeted to arbitrary mRNAs or to other nucleic acids (e.g., for self-assembly of RNA/DNA nanotechnological objects or devices). The fingerloop can take one of two main configurations, either with antisense sequences in the descending strand or the ascending strand of the helix as well as the loop region. These configurations can be reversed, swapped, or duplicated. For example, both stem-loops could use an ascending strand plus the loop sequence, leading to different combinations of fingerloops with varying efficacies against target mRNA gene expression, self-assembly, etc. The stem region is more tolerant to these mismatches in target sequences if the loop region is a perfect match. In addition, by increasing the intrinsic stem stability to make the stem structure longer, the off-target filtering efficiency can be improved. In other embodiments, fingerloop filtering is done in combination with toehold-sequence filtering. The toehold region is adjacent to the base of the stem.

The chimeric DNA fingerloops are based on the structure of the native DsrA sRNA. The native DsrA sRNA contains gaps in its antisense sequence against two E. coli mRNA targets, rpoS and hns. The function of these gaps is not known, as engineered mismatches continue to function. The native DsrA contains both toehold-like and fingerloop-like antisense motifs which may tolerate rather than filter mismatches in loop antisense sequences. In the detection technologies described herein, in some embodiments, the gaps accelerate or modulate the kinetics of annealing.

The use of chimeric DNAs containing two fingerloops, and the capacity to target more than two mRNAs, shows that synthetic multi-fingerloop chimeric DNAs can be built for targeting and coordinating expression from larger numbers of genes. In some embodiments, 4-fingerloop chimeric DNAs using the genetic system disclosed herein with fluorescent reporters are used. In some embodiments, the buk′ and hydA′ fingerloops are built onto the 5′-end of native DsrA structure to create a chimeric DsrA-like DNA with 4-mRNA targeting (for example, rpoS, hns, buk, hydA).

Applications of these multi-acting chimeric DNAs include tuning pathway gene expression for metabolic engineering of strains, for example coordinating multiple mRNAs in single or multiple/different pathways, for increasing fermentation product yields, fermentation selectivity, minimizing toxicity, and/or balancing cellular health and growth rates. Further, combinatorial knocking-down of multiple mRNAs (including essential genes that cannot be conventionally knocked out) can be used, for example, in screening drug targets in pathogens, for determining metabolic flux parameters in bacterial strains for metabolic engineering, and for producing probiotic or commensal bacterial strains.

There is a relationship between the efficacy of fingerloop gene-repression activity in vivo and the free energy parameters of both the fingerloop (self-pairing) and the fingerloop-target interactions. The thermodynamic driving force for pairing of fingerloop and target is related (or proportional) to the differences in stability of the self-paired structure of the fingerloop, the self-paired structure of the target mRNA, and the final stability of the paired complex. Factors that can contribute to stability of various forms are: (a) The length and strength of the fingerloop stem sequence, including its base pair composition; (b) the existence, if any, of mismatches, bulges, and other non-canonical base pairs in the stem; (c) the length, sequence composition and structure, if any, of both the antisense and target regions; (d) the length and strength of the duplex formed between the fingerloop and its target; (e) the length, strength and composition of the loop region of the fingerloop; (f) the fraction of the loop sequence that contains antisense sequences, since extra sequence could be included in this loop outside the antisense sequence; (g) the length, strength and sequence composition of a toehold sequence adjacent to the stem, and the complex formed between the toehold and the target; (h) the type, location and number of mismatches or bulged nucleotides formed between the chimeric DNA and its target; (i) the cooperativity, if any, between the toehold: target and fingerloop: target interactions.

In some embodiments, fingerloops can be delivered to a cell by leveraging existing cellular uptake means such as endocytosis, and can be carried on a nanoparticle made of RNA, DNA or other material.

In other embodiments, fingerloops can be packaged in a phage or viral capsid, envelope, liposome, or other delivery matrix, and can thereby be taken up into a cell.

In some embodiments, the chimeric DNA can comprise at least one chemically modified nucleotide. Various chemically modified nucleotides are known in the art, for example, see WO/2018/009822.

In one embodiment, the at least one chemically modified nucleotide is a chemically modified ribose. In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is selected from 2′-O-methyl (2′-O-Me) or 2′-Fluoro (2′-F). In one embodiment, the chemically modified ribose is 2′-O-methyl (2′-O-Me). In one embodiment, the chemically modified ribose is 2′-Fluoro (2′-F).

In one embodiment, the at least one chemically modified nucleotide is a chemically modified nucleobase. In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In one embodiment, the at least one chemically modified nucleotide is a chemically modified phosphodiester linkage. In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), 2′,5′-phosphodiester (2′,5′-PO) or morpholino (phosphorodiamidate morpholino oligomer). In one embodiment, the chemically modified phosphodiester linkage is phosphorothioate.

Other modified nucleoside analogues can be found in US20080261823 titled “Fluorescent Nucleoside Analogs That Mimic Naturally Occurring Nucleosides” by inventor Yitzhak Tor.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Functional DNA “Fingerloops” for Nucleic Acid-Targeting and Detection Technologies

RNA and/or DNA sequences can be synthesized to self-assemble into a nucleic acid helix according to well-understood nucleotide base-pairing rules. However, mismatched base pairs can be tolerated to some extent depending on the stabilizing effect of flanking helix structures, leading to a “bulged” helix containing a mismatch. Consequences of sequence-mismatched “off target” binding in vivo include incorrect mRNA targeting by regulatory RNAs and may be associated with disease states. For in vitro diagnostics that use nucleic acid base pairing as the basis of detection, incorrect pairing with a target can lead to false positive diagnostic results.

A structured antisense molecule was designed that appears to reject mismatches in as many as 18 nt of contiguous probe: target base pairing. This structural design was informed by studies of a small regulatory RNA in E. coli.^{2,4} Many sRNAs have sequences that pair by antisense base pairing interactions with mRNAs. Typically, the regions in native sRNAs responsible for antisense-targeted mRNA pairing are single-stranded and largely unstructured, which were predicted to facilitate pairing in trans.⁹ In contrast, the E. coli DsrA sRNA contains two stem-loops with antisense sequences located in the loop, with the antisense sequences continuing into one or the other strand of the double-stranded stem.^{4,11} These antisense sequences in native DsrA pair with two different E. coli native mRNAs and regulate their translation.^{2,11} The secondary structure of DsrA is conserved across 3 bacterial species,² thus it seems likely that this secondary structure encodes a function. This structural motif was abstracted to design retargeted-antisense sRNA variants that effectively target two distinct, novel transcripts orthogonal to E. coli in vivo.^{12,13} The structured stem-loop antisense motif was named a “fingerloop,”^{12,13} in comparison to the “toehold” motif used for DNA nanotechnology strand-displacement networks.^{7,10} It was hypothesized that the intra-strand partner to the stem portion of the fingerloop antisense sequence in the helix (the stem-complement strand) acted as a cis-acting competitor to diminish target pairing in trans with a mismatched target strand. In unpublished work, a “mismatch exclusion” principle at work in mutant sRNA variants and reporter genes with mismatches in the antisense components was demonstrated. Further unpublished work has demonstrated the mismatch exclusion principle in vitro using DNA fingerloop “probe” molecules and single-stranded DNA “targets”.^{14, 15, 16}

Structured fingerloop antisense probes were experimentally compared to unstructured single-stranded detection probes for their ability to exclude mismatches in a target molecule, either an mRNA-fluorescent reporter gene in vivo or an unstructured DNA oligo in vitro. Several DNA variants of the sequences used in vivo incorporated into engineered sRNAs were demonstrated to exclude mismatches were prepared and tested. Some of these structured DNA fingerloop constructs can exclude single-nucleotide (point) target mutants from pairing at all 18 antisense positions.

These fingerloops potentially have optimized loop sizes, stem lengths and thermodynamic stabilities. However, it is unclear if this stringency necessarily holds true for all sequences, nor if the optimum for any given sequence would use the same size and stability parameters. It may be that each sequence will need some combination of optimized structure and thermodynamic stability parameters for mismatch exclusion at all desired positions. For a contiguous target of 18 nt, this level of selectivity would mean that I sequence could in principle be detected from a pool of approximately (4¹⁸−1) mismatched sequences (1 in 68.7 billion). If this level of selectivity could be achieved for an arbitrary sequence, this antisense technology would lend itself to very precise diagnostics involving nucleic acid detection by base pairing.

Currently studies are performed on whether the mismatch exclusion principle is sequence-universal for fingerloop target sequences, or whether there are constraints on the sequences that can be detected with single-nucleotide specificity. It is hypothesize that individual sequences have optimal loop, antisense, and stem stability components required for mismatch exclusion behavior, and that a departure from that optimum reduces the number of antisense base pairs capable of mismatch exclusion. It may be that generating, testing and studying many such sequences that can exclude mismatches will allow us to determine the key requirements of mismatch exclusion for any given sequence. Ideally, these parameters could be analyzed in silico via a free-energy computation8 using a given sequence folded into different fingerloop sizes and stabilities, and taking into account the calculated free energy of stability of the fingerloop probe: target complex.

The approach has been as follows: perform tests in vivo (sRNA: reporter mRNA) and in vitro (DNA oligo annealing and gel-shift) to systematically vary loop, stem, antisense length, and thermodynamic parameters relevant to correct target recognition with a single-nucleotide threshold of mismatch exclusion.¹⁴ Fluors and quenchers were also used to design “fingerloop beacons”, similar to shared-stem molecular beacons^{1,3,5} but with distinct design parameters. In particular, the fingerloops have longer and more stable helical stems than molecular beacon probes.^{6,15,16} Taken together these experiments demonstrated that (a) the length of the stem and the size of the loop are important determinants of fingerloop mismatch-exclusion functions for a given sequence in vivo and in vitro; (b) the RNA fingerloops in vivo and the DNA fingerloops in vitro are both capable of excluding mismatches, although the mismatches are largely excluded from the loop region only in vivo, possibly due to protein effects and possibly due to the absence of tuner sequences in vivo. (c) Fingerloop beacon assays suggest the base of the stem helix opens when binding an exact-match target and when binding a mismatched target that is not successfully excluded.¹⁴ Models of nucleation of DNA toehold base pairing¹⁰ suggest the initial in trans base pairing (seed) will begin in the loop region and will be slow and rate-limiting. In this model the seed pairing reaction is followed by a more rapid strand-displacement/branch migration event. The base-paired complex of probe and target is thermodynamically stabilized relative to the probe and target because the complex formation reaction increases the total number of base pairs as it displaces the stem-complement strand, increasing product-complex stability. Both the loop and stem regions have distinct contributions to filtering out mismatches and excluding them from complex formation, possibly by different mechanisms.

Further gel-shift experiment were performed. Both linearized and structured (fingerloop) antisense probes were built using novel randomized 18-mer DNA sequences as well as the complementary “sense” target strands of DNA. Oligos were then designed and synthesized and were tested in lab class by gel-shift assays with SYBR Gold gel staining for visualization. This exercise has investigated mismatch exclusion and role of the two topologies of fingerloops: ascending fingerloops (5′-strand plus loop antisense) versus descending fingerloops (loop plus 3′-strand antisense); the role of extended stems containing “nonsense tuner” sequences to increase stability (decrease free energy) and buffer the annealing reaction against temperature changes; and the role of loop size in creating fingerloop probes capable of mismatch exclusion. The role of sequence is less clear although it appears that G: T mismatches in DNA are tolerated and anticipates G:U RNA pair mismatch tolerance, also seen in molecular beacons.³ Removal of G:T or G:U pairs will decrease the selectivity of mismatch exclusion (below 1 in 418-1 for an 18 nt antisense region). To improve assay throughput fluorescent probe design similar in function to molecular beacons will be designed, tested, and iterated. These fingerloop beacons can be used to scale the analysis of individual annealing reactions in 96-well or 384-well test platforms to supplement gel shift assays. Both gels and the in vivo growth and fluorescence assays are time-consuming to construct and assay, so the plan is to synthesize RNAs or derivatives (e.g., 2′-F RNA) to test variant RNA molecules in vitro via fluorescence.

However, the DNA oligos constitute a cheap and flexible basis for probe design and assay. A comparison of equivalent antisense sequences were fashioned into either fingerloop probes or molecular beacon-like probes, albeit tested via gel shift assay. Each of several probes were tested for their capacity to exclude a single mismatched nucleotide in eighteen point-mutant variants of their corresponding 18-mer targets, with each mismatch designed to disrupt a single base pair along the 18-mer.

Contrary to expectations the molecular beacon analogs (both conventional and shared-stem beacons) tolerated mismatches at almost all positions when prepared from random unstructured sequences, whereas some of the fingerloop sequences rejected mismatches to the same targets. Although mismatch exclusion was claimed for molecular beacon assays performed in solution phase,³ it is possible the gel assays are more stringent and may stabilize mismatches within the polyacrylamide gel matrix. Further studies using fingerloop beacon assays in solution phase may reveal a more widespread mismatch exclusion in diverse fingerloop sequences.

Fluorescent readout of successfully designed probes with stringent mismatch-exclusion properties will serve as the basis for future point-of-care diagnostics. Such a test can be administered by nearly anyone, similar in flexibility and availability to home pregnancy tests but potentially at very low cost.

### Results

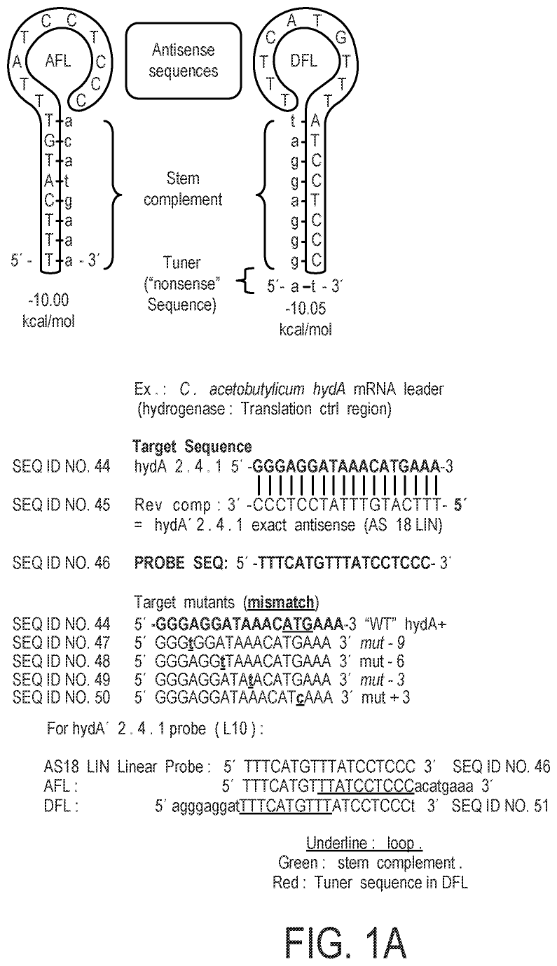

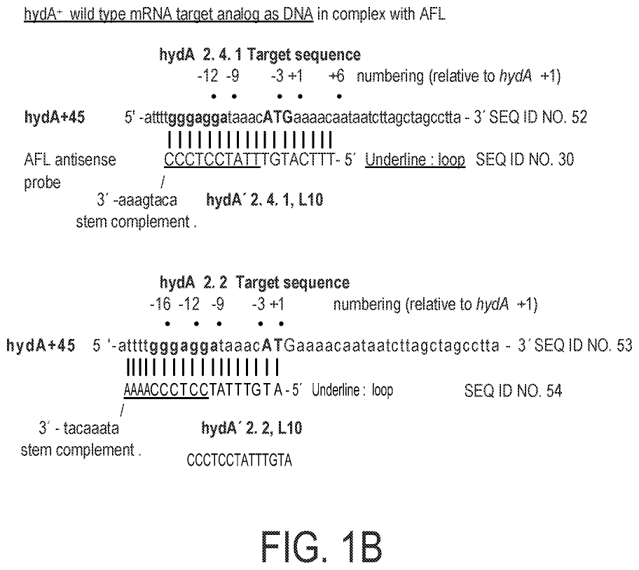

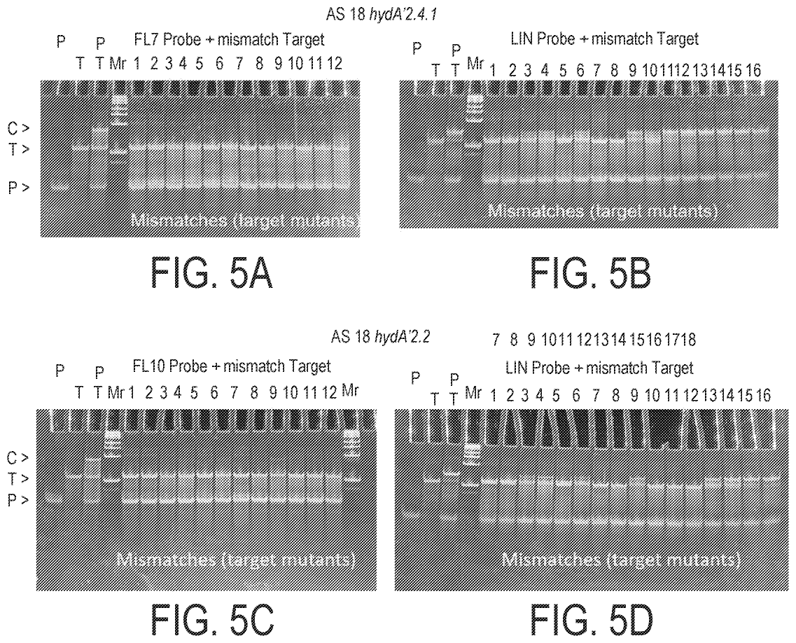

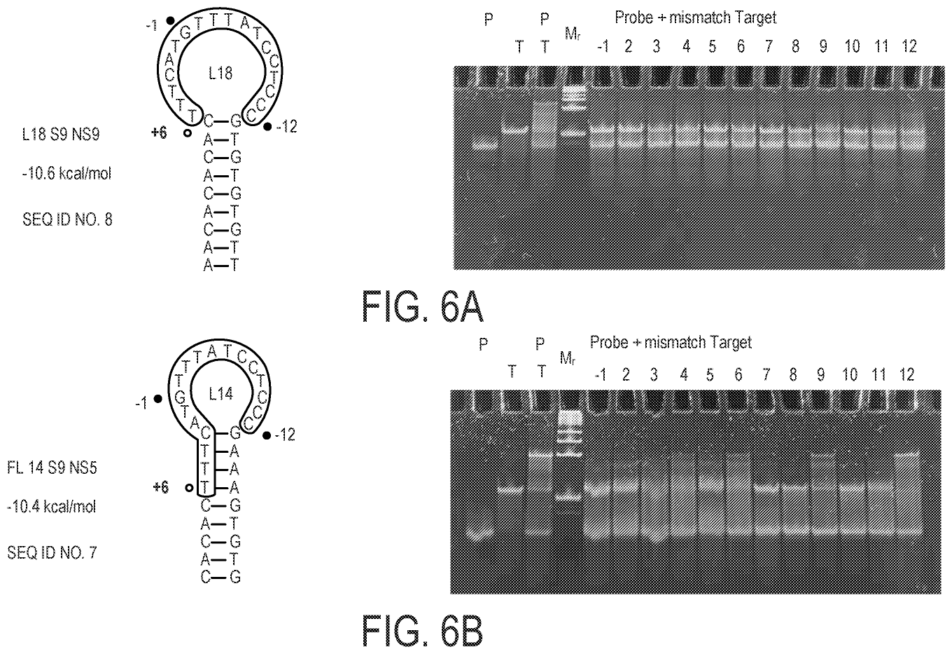

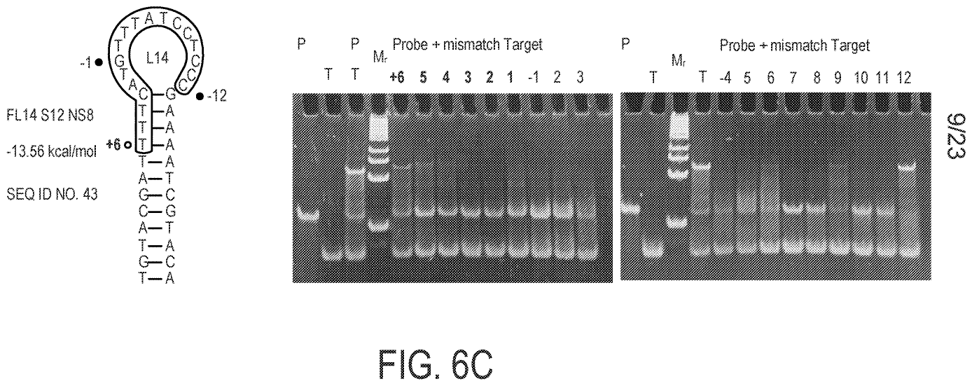

An illustration of the two configurations of antisense sequence on fingerloop structures including antisense sequences, ascending or descending fingerloop is shown in FIG. 1. In addition to the central loop component of the antisense sequence the stem-resident antisense sequence can be in the 5′-portion (AFL) or the 3′-portion (DFL) of the stem. If the exact same 18 nt of antisense sequence is used in both probes, the thermodynamic stability typically changes unless the nucleotide % GC or melting temperature (Tm) of the loop region and stem-region sequences are identical (for example, a GC-rich loop sequence in AFL becomes a GC-rich stem sequence in the DFL configuration). The loop component contributes some free energy but the bulk of the fingerloop's structural stability is contributed by the stem. In the case shown, the DFL probe required 1 bp of nonsense tuner to match the AFL stability so that comparisons between the two forms are calculated to be based on near-equal relative free energies. All free energy and structural calculations are done in NUPACK (www.nupack.org) (J. N. Zadeh, et al., J Comput Chem, 32:170-173, 2011). In FIG. 1B we see an illustration of two models of fingerloop: target complex that compares the location of two probes mentioned in FIGS. 5-6 (hydA2.4.1 and hydA′2.2). These fingerloops were originally tested in sRNA variants and demonstrated to decrease translation from a hydA-mCherry reporter gene (Lahiry et al. 2017). These sequences were subsequently synthesized as DNA oligos for further testing and optimization. The map location of mutations in hydA are given above the mRNA analog (DNA oligo). The numbering of positions is relative to the start codon (+1) and covers the range from −12 to +6 (hydA2.4.1) or −16 to +2 (hydA2.2). The stem complement sequences are in green; the loop regions are underlined; the target regions in hydA mRNA are in red with the Shine-Dalgarno ribosome binding site and start codon (+1) in bold. Both fingerloops are depicted here in the AFL configuration.

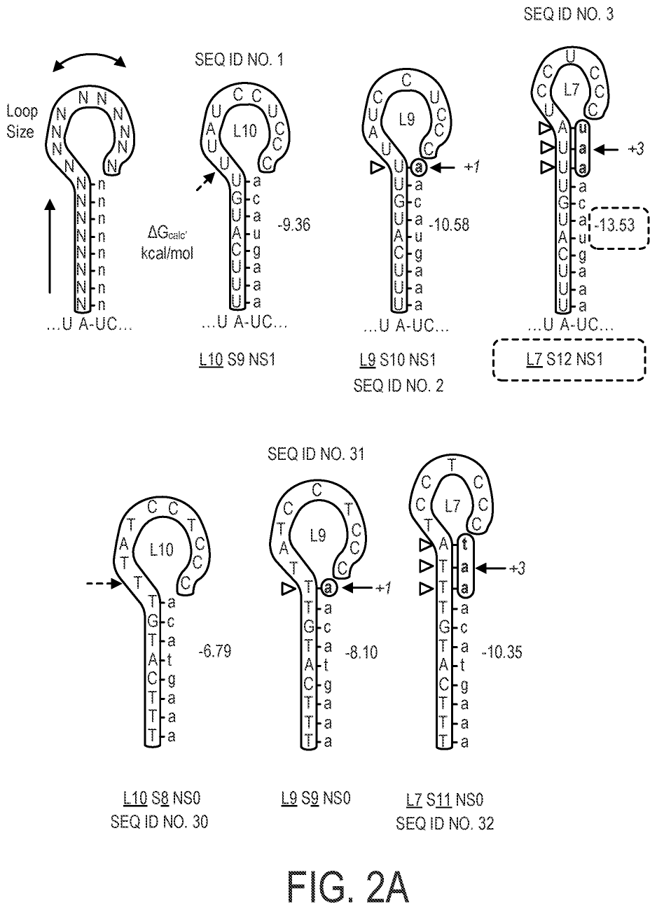

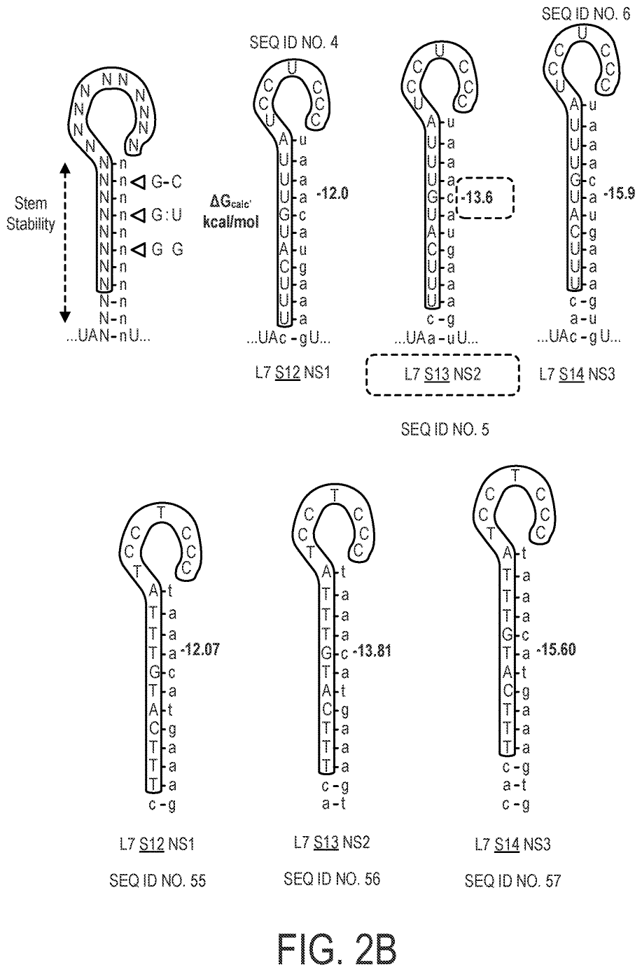

FIG. 2A-2B demonstrates that, while not changing the contiguous antisense sequence (white letters on a black background), both the loop and stem lengths can be modulated to optimize the probe: target interaction. The criterion for optimization of a fingerloop probe is that it (a) binds the exact-match target, and (b) rejects mismatched targets, with an exclusion threshold of 1 point mutation in 18 nt of antisense probe sequence (theoretical selectivity of 1 in ˜4¹⁸ 18-mers). These structural stem and loop perturbations modulate thermodynamic structural stability; insufficiently stable probes cannot exclude mismatches, and excessively stable probes cannot bind exact-match targets. Probes are designed, synthesized as DNA oligonucleotides or RNA oligonucleotides, and assayed by gel shift or fluorescent methods in binding targets and mismatches. (FIG. 2A) variation of loop length is achieved by adding single or multiple stem-complement nucleotides at the top of the stem on the (AFL) 3′-stem strand. Addition of a nucleotide that pairs with the first or the first three loop antisense nucleotides (+1 and +3, respectively, are shown) reduce the loop size and increase the length of the stem. (FIG. 2B) sequences are added at the base of the stem to increase the thermodynamic stability of the stem without modifying the loop or antisense sequence. These “nonsense tuner” sequences do not match the target and thus do not extend the antisense base pairing, but the thermodynamic stability increases with longer tuners. Thus, all these probes retain the exact antisense sequence located in subtly different places on the fingerloop stem-loop. When adjusted for stem stabilities the probes can be compared on the basis of equal free energies of stability to determine loop-specific or stem-specific contributions to fingerloop binding activity and mismatch exclusion activity.

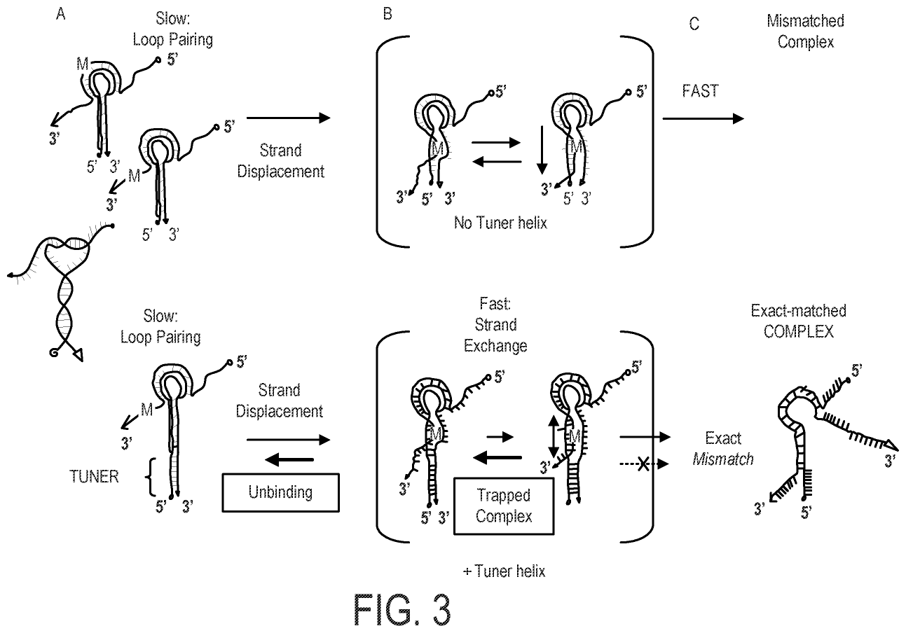

A model for loop and stem mismatch exclusion is shown in FIG. 3. FIG. 3 shows secondary structure diagrams depicting fingerloop stem-loops binding linear target analogs. A 2D/3D representation of the loop: target complex is also shown. (A) “slow: Loop pairing”, the initial nucleation of base pairing is thought to proceed by a loop: target interaction because the stem-antisense sequence is less available to pair while sequestered in the fingerloop stem, bound to the stem-complement “internal competitor” strand. Studies using toehold sequences, single-stranded (not loop) antisense sequences that extend into a double-stranded base paired portion, predict that the nucleation step is slow, followed by a faster branch migration/strand exchange step (B) that may resolve into a full exact matched or mismatched complex (C). (A), mismatches in the target that correspond to positions in the loop are excluded by intrinsic loop-mismatch properties, but stem-mismatched targets pair in the loop and proceed to a strand invasion/branch migration. (B), intermediates that have bridged the thermodynamic barrier for strand invasion and are displacing the stem-complement are seen. However, when comparing the top and bottom schemes, the nonsense tuner and its stem-complement strand does not pair with the remaining portion of the target, preventing unwinding of the helix. Accordingly, the branch migration complex halts, and no further strand invasion is possible. If the target is mismatched in the stem, the reverse reaction may be favorable and the mismatched target can fall off (red arrows, leftward). If the target is an exact match, the pairing becomes sufficiently stable to form a full complex, a separate step is the opening of the fingerloop stem helix. In the absence of tuners, the bottom of the helix opens as the antisense target extends to the 5′-end of the fingerloop. If the nonsense tuner is very stable, even exact match targets do not form final complexes, and the reverse reaction (pairing of the tuners and exclusion of the target) is favorable. It is not clear whether strand separation is required for stable complex formation or mismatch rejection. If the reverse reaction is favored, depending on the relative stability of the transition state the target: probe complex may become trapped in 1 or more intermediates that are nonproductive for full complex formation. Consistent with this model, when the tuners are so stable that exact-match complexes do not fully form, species in the gel resembling a triplet band migrating faster than the complex are seen. These triplets may represent loop-target interactions that do not progress into the stem helix, or various complexes that form and release the branch migration junction, neither of which resolve into a species migrating as a full complex on a gel.

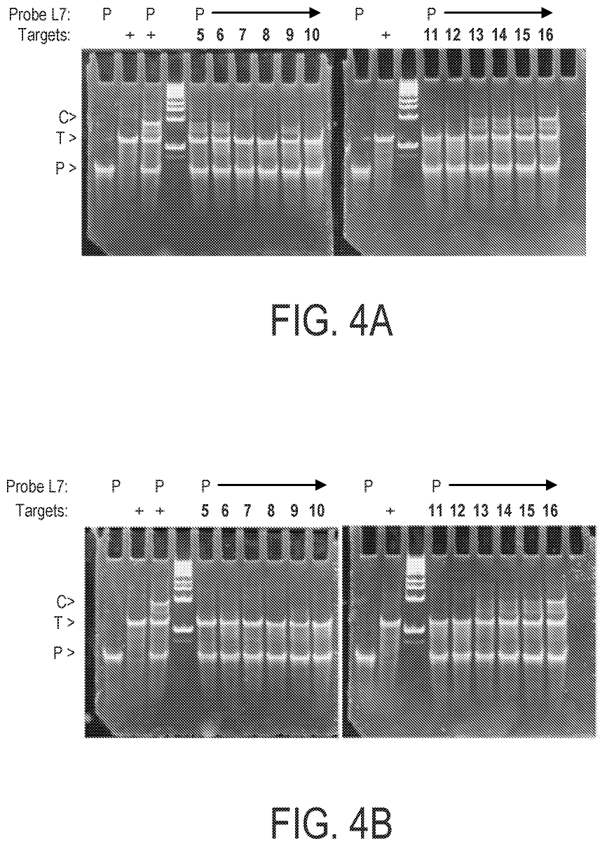

FIG. 4A-4B images show contrasting cold/room temperature annealing experiments for the fingerloops (annealing and gels temperatures) using the fingerloops that do not have tuners. The images demonstrate improved filtering at elevated temperature. Also, leakiness (defined as formation of a population of mismatched complexes) is much reduced but not eliminated, which makes it a good example for how temperature alone is a difficult parameter for optimization. Since temperature impacts the free energy of structural stability of the probe: target complex, altering stem stability with the tuner was a more controllable parameter and more robust to temperature changes during the assay.

FIG. 5A-5D shows that the mismatch rejection activity of fingerloops is inherent to the structure. (FIGS. 5A and SC) the positive control (exact match target, second vs third lane) shows that under these conditions (constant 23° C. incubation for annealing and electrophoresis) the correct match will generate a probe: target complex, whereas any point mutant across 12 nucleotides of antisense sequence will fail to form a complex. (FIGS. 5B and 5D) a linear target reveals complex formation between the linearized (not fingerloop) probe and the same target mutants. The absence of mismatch filtering/mismatch proofreading is shown as complexes formed (e.g.) in lanes 3, 4, 6, and 9. Complexes in lanes 13-16 are formed with targets whose mutations lie outside the antisense region of the probe, and are present on the second gel only.

FIG. 6A-6B shows that in a room-temperature annealing experiment with temperature-stabilized gel system (23° C.) that an L18 end-loop with a long stem and antisense in the loop relies on loop mismatch exclusion, a known property of (e.g.) molecular beacons, but here the stem is long enough to exclude mismatches at all but the last position. However, comparison of this effect to a near-relative fingerloop L14, FIG. 6B demonstrates some leakiness is occurring in the L14. The free energies are comparable to the in vivo system, but successful tuners convey improved stem stability and improved mismatch rejection.

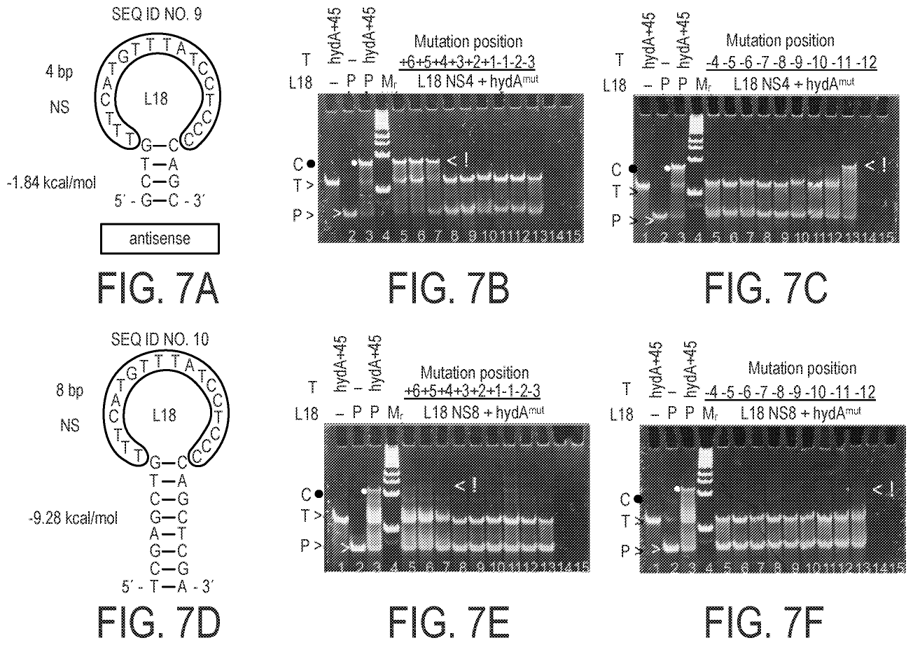

FIGS. 7A-7F show images of two beacon analogs (L18) with different size stems. The two stems are designed as nonsense sequences; all the bases in the stem including topmost base pair at the edge of the loop are nonsense sequence. Only the loop nucleotides are antisense. A series of these molecules were designed with increasing-length nonsense stems and lower free energy (greater stability) as they go longer. The comparison suggests that increasing the length of the nonsense tuner eventually enhances mismatch proofreading during target binding.

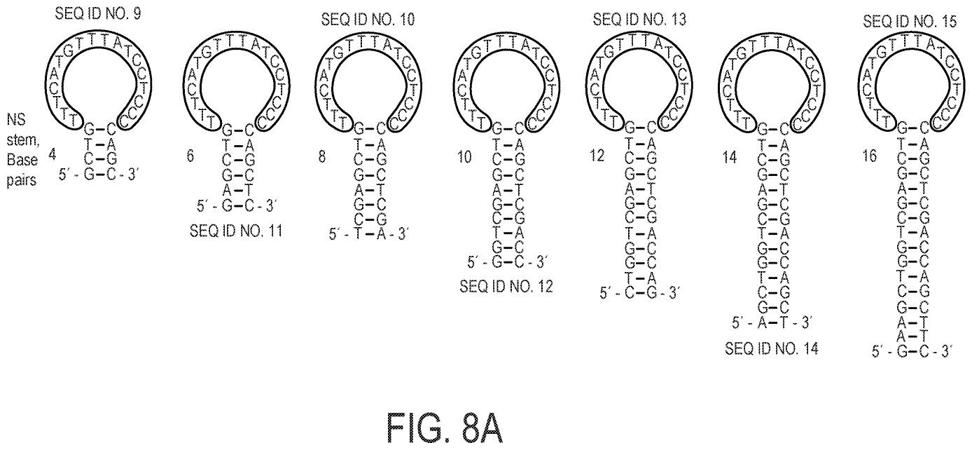

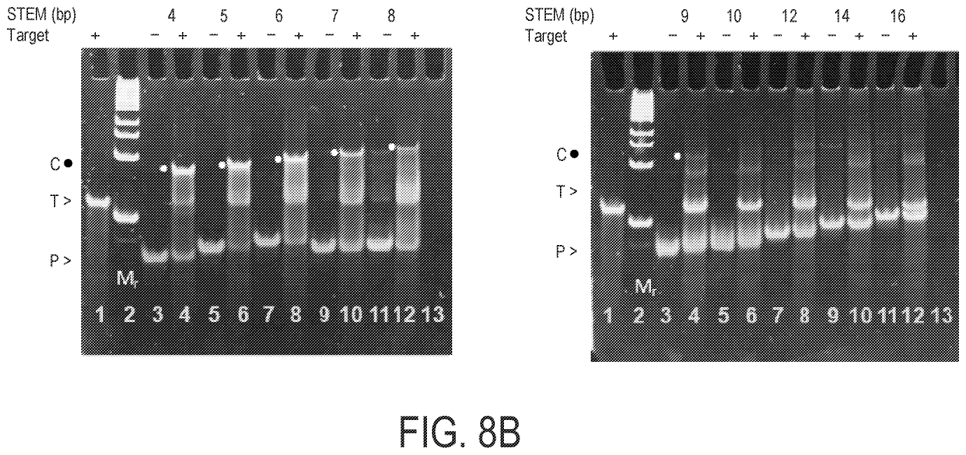

FIGS. 8A-8B show that the beacon becomes largely incapable of binding the exact-match control at 9 bp stem and longer. It is possible the stem is no longer opening because the target: probe sense-antisense interaction needs to form a double helix and the nonsense stem helix is sterically blocking helix formation. Alternatively, the full complex is not formed unless the stem opens (separates), and if there is no target: probe (sense: antisense) interaction in the stem, the stem will remain closed if it is greater than some threshold of free energy.

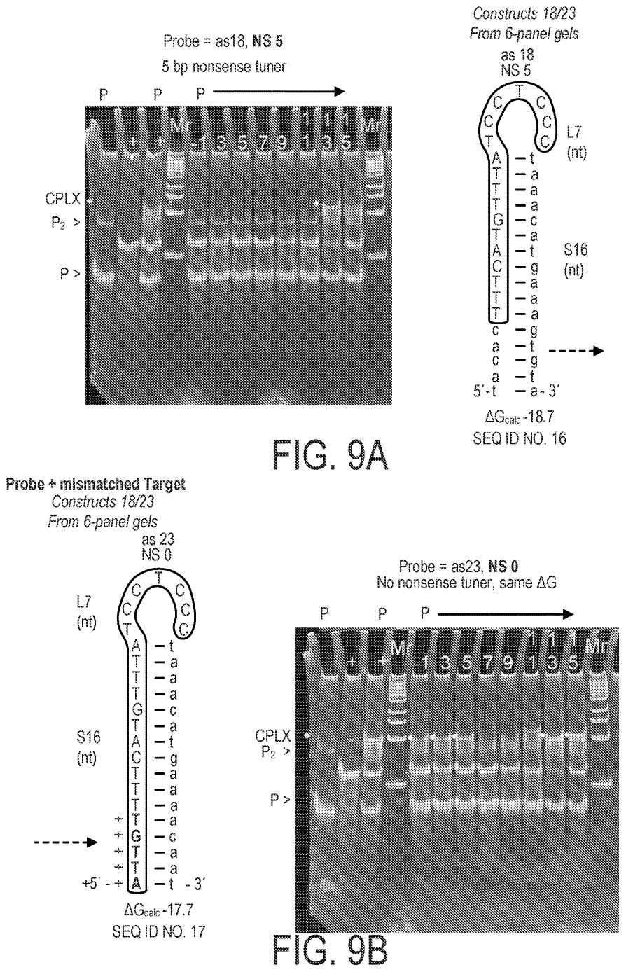

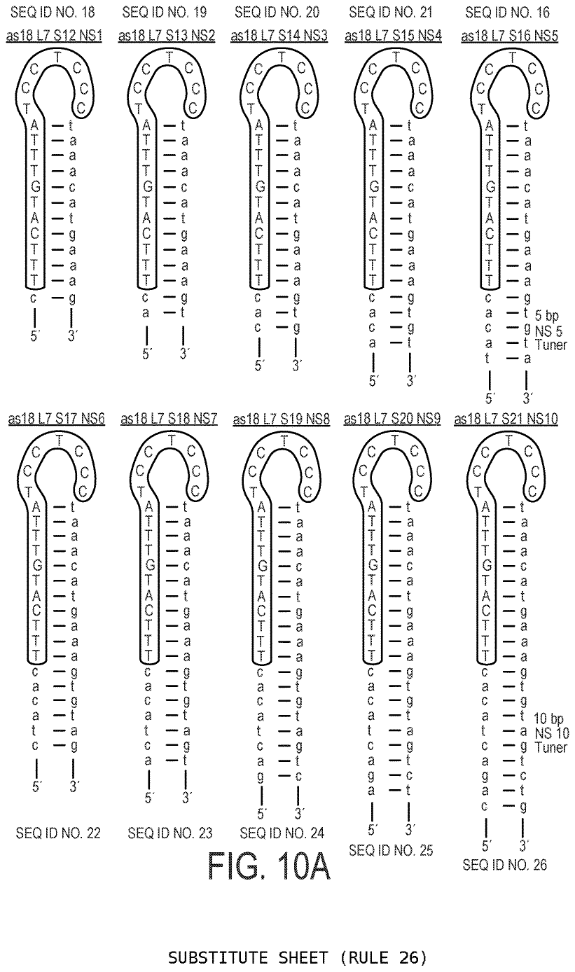

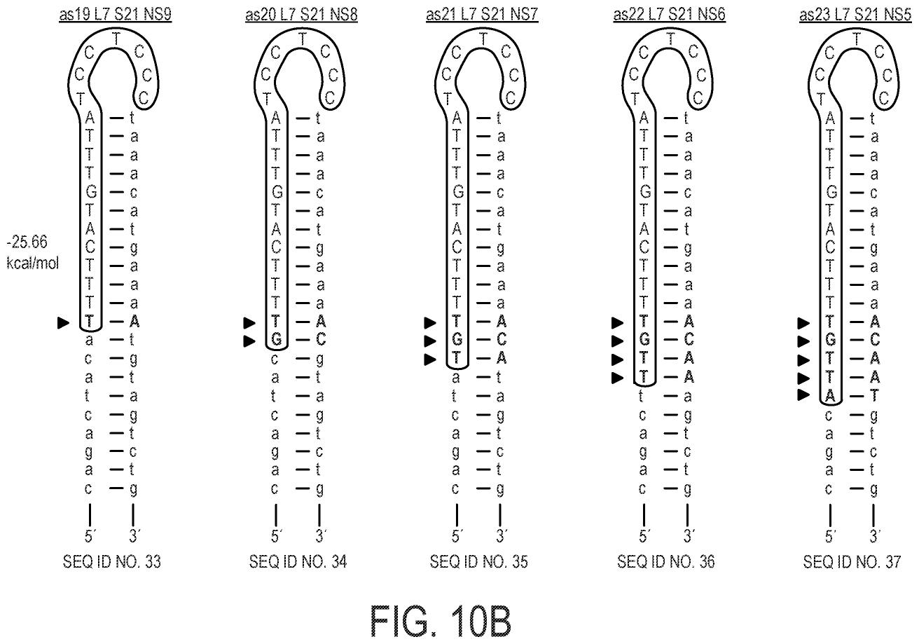

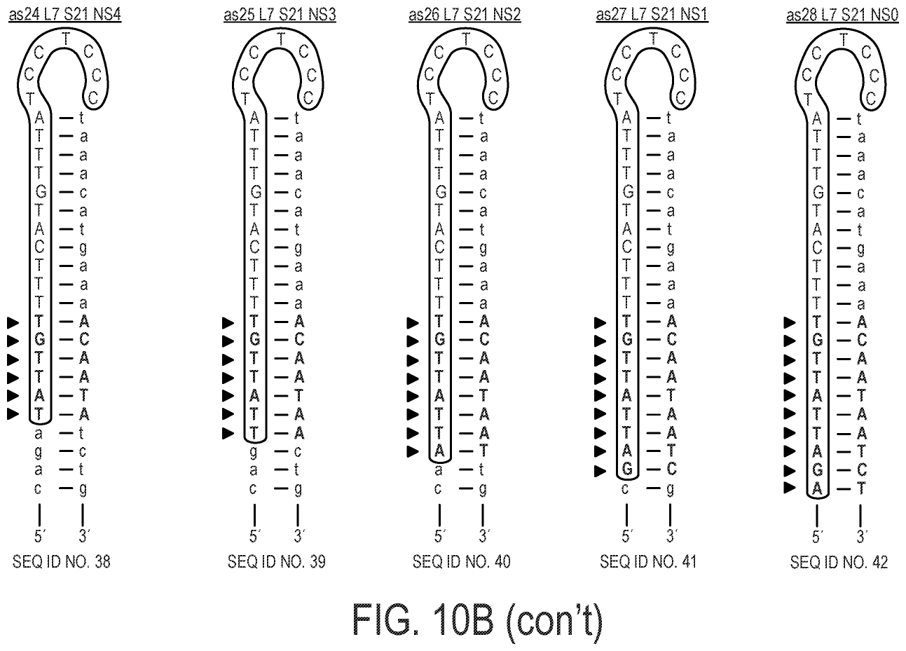

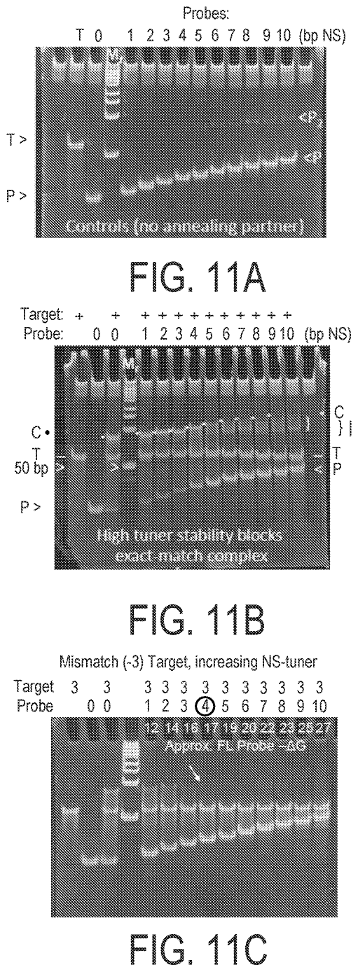

FIG. 9A-9B demonstrates that a robust tuner at the bottom an L7 probe will enable mismatch rejection at multiple positions over the stem and loop. When the tuner is removed by increasing the antisense from 18-23 nt, the probe loses its capacity for proofreading and rejecting mismatches. FIG. 9A-9B demonstrates that a process in the stem, involving the tuner, is essential to enhancing mismatch rejection as shown in FIGS. 10-11.

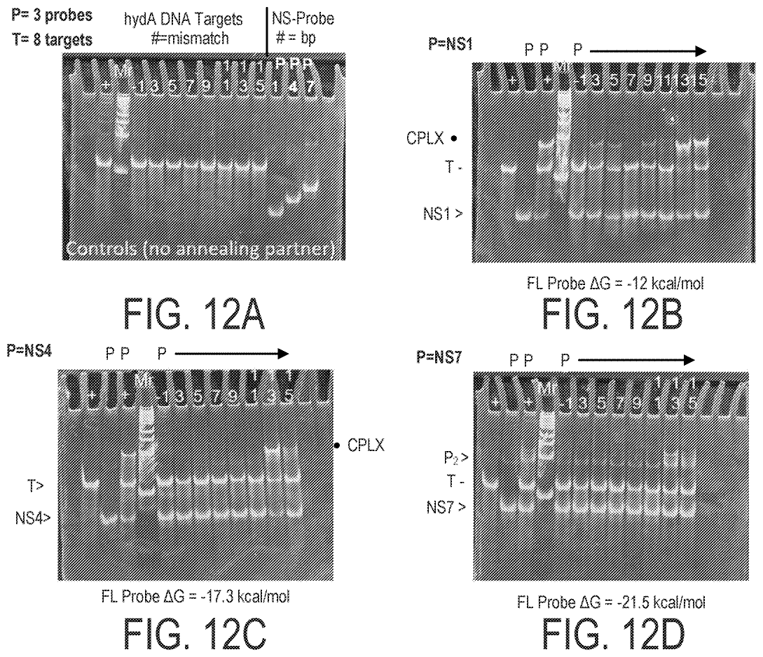

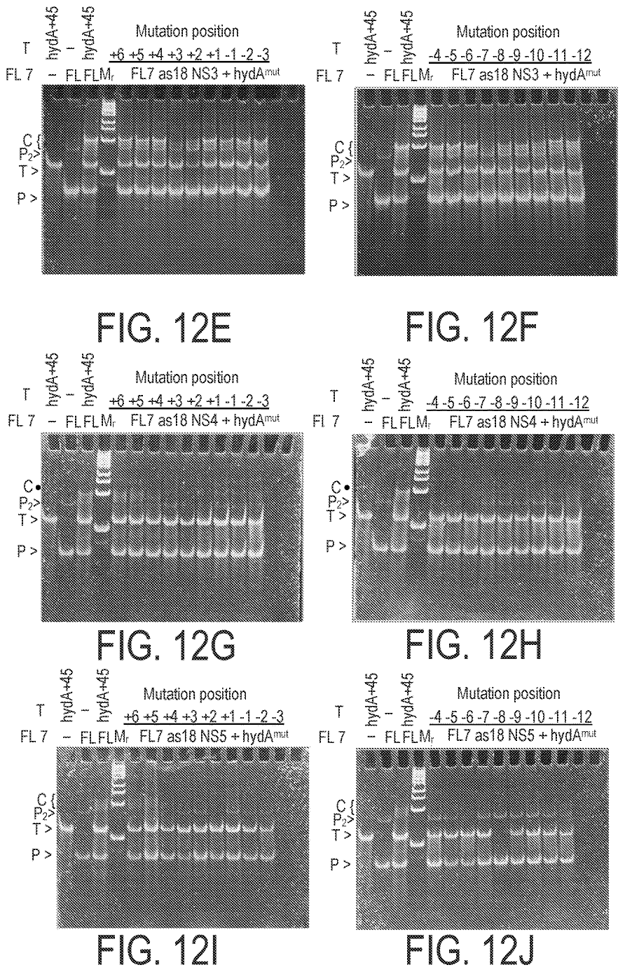

FIG. 10A shows a 7-nt loop fingerloop probe series related by an increasing number of base pairs at the bottom of the stem (“nonsense tuners”). These are “nonsense sequences” that cannot form Watson-Crick base pairs with the target and are unrelated to either the target or the antisense regions of the probe (white letters on a blue background). As these tuners are steadily increased in length the free energy of stability of the stem is increased, although the target sequence remains the same length (18 nt). On the following gels (FIGS. 11-12) these probes were tested against both exact-match targets and their point mutant derivatives (mismatched targets).

Study of probes (FIG. 10A) with different lengths of “nonsense (NS) tuner” at base and their capacity to bind several targets are shown in FIGS. 11A-11C. (FIG. 11A), the individual probes (NS1-NS10) without annealing partner are seen. At the higher end of stability (lanes ˜5-10), dimer formation (P2, probe dimer) is seen. As control reactions, the hydA+45 target (T) and the unmodified fingerloop (NS 0) are on the leftmost side with NEB low-ladder marker (lowest band is 50 bp dsDNA). (FIG. 11B), annealing controls (target and probe NS0 alone and together) next to the ladder marker are seen. The lanes from left to right are in order of increasing stability of the fingerloop probes as they are annealed with the exact-match target. Complexes are formed readily when the fingerloop has 0 to 4 base pairs of nonsense sequence at the bottom of the stem, covering a range of increase in structural stability from ˜12 kcal/mol at 0 nt tuner to −17 kcal/mol for the 4 bp tuner). As the stem stability increases, shifted species are seen that are more disperse and fainter, corresponding perhaps to probe: target complexes that bind only in the loop, considered as potential intermediates in the binding reaction. The stems are too stable to admit an invading exact-match strand and cannot be used to check for mismatches (negative result is uninformative). Establishing this endpoint of activity is important for defining the optimal fingerloop stem stability for the target sequence. (FIG. 11C) the −3 mismatch binds the NS0 control but cannot bind the NS 2.

In the experiment depicted in FIG. 12A-12D, individual fingerloops and mutant targets are shown alone in the gel (FIG. 12A) and then fingerloop probes with nonsense length NS1, NS4, and NS7 are each combined with an exact-match control or a panel of point-mutant targets, (FIGS. 12B-12D, respectively) Formation of complexes between the NS1 probe and mismatched targets (FIG. 12B), target mutations 3, 5, 9, and 11) indicate the fingerloop is too unstable to reject mismatches. In the NS4 probe (FIG. 12C), the mismatches do not form stable complexes which are absent from the gel. Three positive controls (exact-match probe, third lane; mutations 13 and 15) demonstrate the correct gel position of the slow-moving complex (uppermost band in each lane). In the NS7 panel (FIG. 12D), a series of bands are seen in the mutant lanes that are not full complexes. These bands correspond to the probe dimer (see band index, left; P2) and indicate the mismatches were filtered out. However, the exact-match probe (lane 3) only binds poorly, indicating that NS7 might be too stable to make a probe because it does not bind enough correct-match target to give a good evaluation of probe efficacy (cf. NS1, NS4). FIGS. 12E-12J tested NS3/NS4/NS5 probes with a full panel (single nucleotide resolution across 18 nt of) target antisense mutations. The results demonstrate the tunability of the system. There is a fairly small range of stem free energies that productively bind the exact match probe but reject mismatches. The base pair composition of the nonsense tuners may be varied to fine-tune the thermodynamic stability, for example by using more or fewer G:C pairs in a given length tuner.

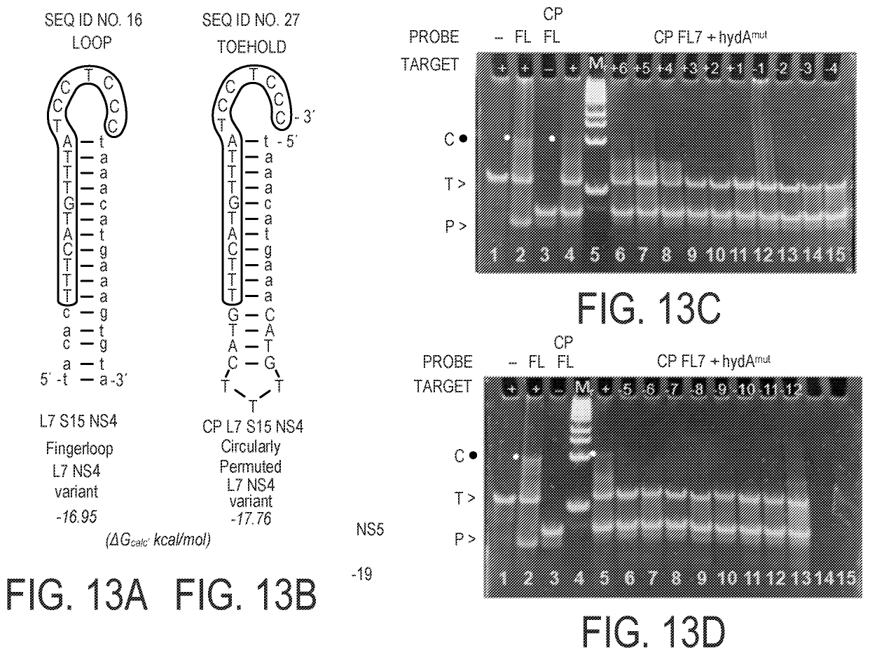

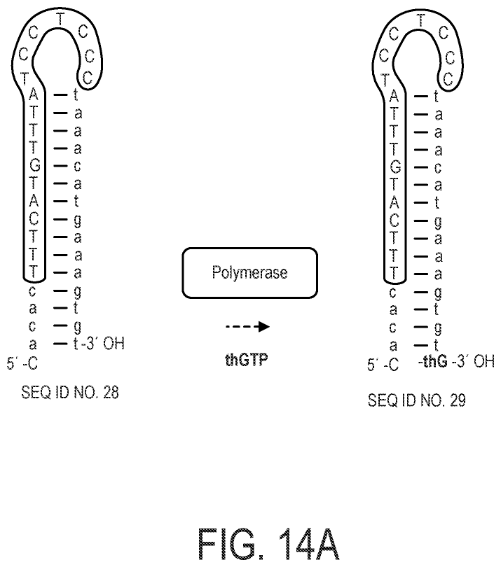

FIG. 13A-13D demonstrates that mismatch rejection does not strictly require the loop, and that in this system (where a nick in the strand opens the loop) steric hindrance of the loop: target complex formation reaction is not required for proofreading. Starting from the L7 S15 NS4 fingerloop (FIG. 13A) a “toehold” variant was created by incorporating a loop at the base and interrupting the loop at the “top” (FIG. 13B), creating a “circularly permuted probe” CP L7). The loop of FIG. 13A is rendered linear in the molecule shown in FIG. 19B, whereas the stem remains mostly the same. The NS4 permuted probe (FIG. 13B-13D) does not strongly bind the exact-match sequence (Lanes 2), so the extent of complex formation and thus the signal range is small. Regardless, the target mismatch point-mutant series do not show evidence of even this small degree of complex formation. The stem, properly tuned, can exclude mismatches so long as the loop region is not sufficient to stabilize a complex between the CP probe and target.

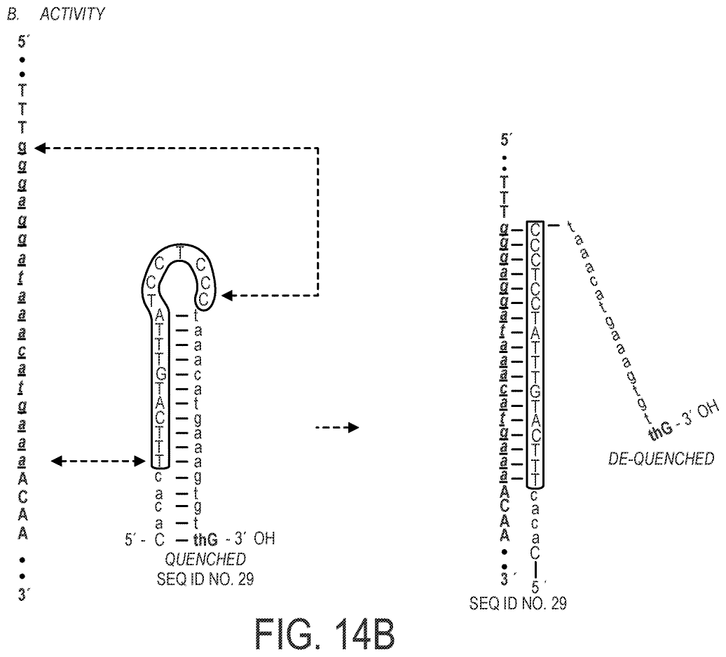

FIG. 14A-B depicts a scheme for fluorescent labeling of fingerloop oligos to mimic the reporter function of molecular beacons. Instead of linking 5′ and 3′ fluors and quenchers, the oligo is synthesized conventionally but lacking the final 3′-nucleotide that is expected to pair with the 5′-most (first) nucleotide. In FIG. 14A the final nucleotide is supplied as a nucleotide triphosphate that is incorporated by extension of the fingerloop using an appropriate polymerase protein; here, a mutant MMuLV RT (Gao, Orlova, et al., PNAS USA 94:407-11 (1997)) for incorporation of thieno-Guanosine (thG) opposite a 5′-cytosine. In FIG. 14B this “self-quenching”fingerloop beacon is shown interacting with its target and forming a complex that releases the stem-complement strand and enables fluorescence of the thG residue.

A panel of fingerloop probes (FIG. 10B) with uniformly long stems were created such that the antisense sequence complementary to the hydA+ target increases over the range of 19-28 bp as the length of the nonsense tuner decreases. This probe set was based on the AS10 probe in the last panel of fingerloop probes (FIG. 10A, probe with longest nonsense tuner). Here, each member of the panel is related by changes to one base pair at a time as they are converted from nonsense to extended antisense sequence. The objective of the experiment is to determine whether mismatch exclusion fails at a particular tuner length as the antisense is increased, but with a broader range of workable tuners than in the previous nonsense-to-antisense conversion panel (FIG. 10A). This experiment uses the panel of probes with longer antisense and tuner set in the previous slide to test whether longer antisense probe (between 19-28 bp of antisense) binds exact match and excludes mismatches positioned in the stem (−3) and loop (−9) of the elongated-stem FL7 constructs depicted in the previous panel. Decreased tuner length as increased antisense length.

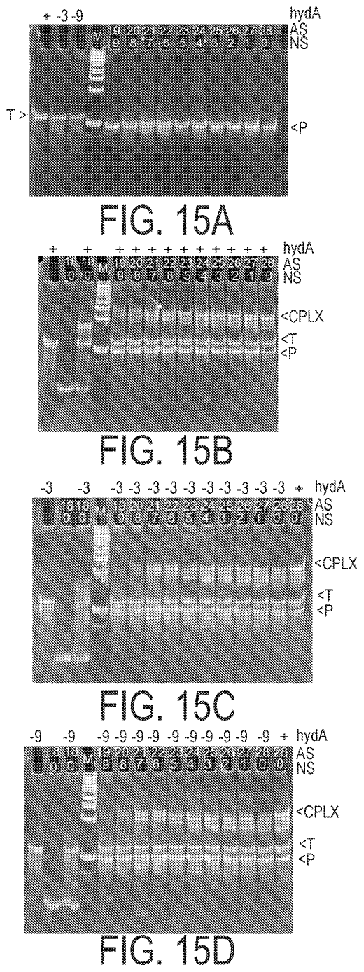

FIG. 15A shows all species in FIG. 10B as well as 3 hydA target variants all without annealing partners; several probes have some minor bands which may be conformers or truncated oligo synthesis products. FIG. 15B shows that exact-match target hydA+ can form complexes with these longer probes, up to about AS22/NS6 (FIG. 15B, yellow arrow); at NS7 and greater the level of complex appears negligible, although intermediates (perhaps loop-target complexes) still form. The positive annealing control probe for hydA+ in lane 3 of FIG. 15B is the AS 18 NS0 probe (L7 AS18 NS0+hydA (+) 45mer). FIG. 15C and FIG. 15D show the long-stem probe series mixed with point-mutant bydA targets that mismatch at positions −3 and −9 (stem and loop respectively in the L7 construct; cf. FIG. 1A-B). The AS18 NS0 control probe binds both −3 or −9 targets poorly (lane 3 in FIGS. 15C and 15D; cf. FIG. 15B lane 3). The last lane of each gel in FIG. 15B-15D is a positive control that shows the migration position of a probe:target complex formed with exact-match target. Both mutations are almost entirely excluded even at the lowest level of nonsense tuner, with even small amounts of complex excluded above probes with AS24 NS4 tuners, despite the longer antisense complementarity. This experiment shows that longer stems containing increased antisense regions still exclude mismatches with tuners of even 1-2 base pairs. The experiment reveals a probe design with theoretically increased selectivity and a potentially broader “sweet spot” for mismatch rejection. The selectivity is theoretically up to about 1 in 422 without taking G: T mismatches into account.

### REFERENCES

1. Tyagi, S. and Kramer, F. R. (1996) Molecular Beacons: Probes that Fluoresce upon Hybridization. Nat Biotechnol 14:303-308.
2. Lease, R. A., Cusick, M. E. and Belfort, M. (1998) Riboregulation in Escherichia coli: DsrA RNA acts by RNA: RNA interactions at multiple loci. Proc. Natl. Acad. Sci. USA 95:12456-12461.
3. Tyagi, S., Bratu, D. P. and Kramer, F. R. (1998) Multicolor molecular beacons for allele discrimination. Nat Biotechnol 16:49-53.
4. Lease, R. A., and Belfort, M. (2000a) A trans-acting RNA as a control switch in Escherichia coli: DsrA modulates function by forming alternative structures. Proc. Natl. Acad. Sci. USA 97:9919-9924.
5. Tsourkas, A., Behlke, M. A., and Bao, G. (2002) Structure-function relationships of shared-stem and conventional molecular beacons. Nucleic Acids Res. 30 (19) 4208-4215.
6. Bao, G., Tsourkas, A., and Xu, Y. (2003) Dual Resonance Energy Transfer Nucleic Acid Probes. US. Patent #US2003/0129611 A1.
7. Zhang, D. Y., and Seelig, G. (2011) Dynamic DNA nanotechnology using strand-displacement reactions. Review; Nat Chem 3:103-113 (DOI: 10.1038/NCHEM.957)
8. Zadeh, J. N., Steenberg, C. D., Bois, J. S., Wolfe, B. R., Pierce, M. B., Khan, A. R., Dirks, R. M., Pierce, N. A. (2011) NUPACK: analysis and design of nucleic acid systems. J Comput Chem 32:170-173
9. Beisel, C. L., Updegrove, T. B., Janson, B. J., and Storz, G. (2012) Multiple factors dictate target selection by Hfq-binding small RNAs. EMBO J. 31:1961-1974.
10. Srinivas, N. Ouldridge, T. E., Šulc, P., Shaeffer, J. M., Yurke, B., Louis, A. A., Doye, J. P. K., Winfree, E. (2013) On the biophysics and kinetics of toehold-mediated DNA strand displacement. Nucleic Acids Res. DOI: 10.1093/nar/gkt801
11. Lalaouna, D., and Massé, E. (2016) The spectrum of activity of the small RNA DsrA, not so narrow after all. Review. Curr. Genet. 62:261:264.
12. Lahiry, A., Stimple, S. D., Wood, D. W., and Lease, R. A. (2017) Retargeting a dual-acting sRNA for multiple mRNA transcript regulation. ACS Synth. Biol. 6:648-658. DOI: 10.1021/acssynbio.6b00261
13. Stimple, S. D., Lahiry, A., Taris, J. E., Wood, D. W. and Lease, R. A. (2018) A Modular Genetic System for High-Throughput Profiling and Engineering of Multi-Target Small RNAs In Arluison, V. and Valverde, C. (Eds.), Bacterial Regulatory RNA: Methods and Protocols. Meth. Mol. Biol. 1737:373-391 (ISBN 978-1-4939-7633-1; doi: 10.1007/978-1-4939-7634-8_21).
14. Lease, R. A., Stimple, S. D., Traverse, E., Tang, S. Y., Barto, J., Konda, L. (unpublished)
15. Lease, R. A., Lahiry, A., Stimple, S. D. (2018) U.S. patent application Ser. No. 15/865,983. ANTISENSE FINGERLOOP RNAS AND USES THEREOF. Patent Pending.
16. Lease, R. A. PCT patent application No. PCT/US2018/042739; WO2019018555A1. (2018) ANTISENSE FINGERLOOP DNAS AND USES THEREOF. Patent Pending.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

SEQUENCES
SEQ ID NO. 1:
5′-AUUUCAUGUUUAUCCUCCCACAUGAAAU-3′
SEQ ID NO. 2:
5′-AUUUCAUGUUUAUCCUCCCAACAUGAAAU-3′
SEQ ID NO. 3:
5′-AUUUCAUGUUUAUCCUCCCUAACAUGAAAU-3′
SEQ ID NO. 4:
S′-CUUUCAUGUUUAUCCUCCCUAACAUGAAAG-3′
SEQ ID NO. 5:
5′-ACUUUCAUGUUUAUCCUCCCUAACAUGAAAGU-3′
SEQ ID NO. 7:
5′-CACACTTTCATGTTTATCCTCCCGAAAGTGTG-3′
SEQ ID NO. 8:
5′-AACACACACTTTCATGTTTATCCTCCCGTGTGTGTT-3′
SEQ ID NO. 9:
5′-GCTGTTTCATGTTTATCCTCCCCAGC-3′
SEQ ID NO. 10:
5′-TCGAGCTGTTTCATGTTTATCCTCCCCAGCTCGA-3′
SEQ ID NO. 11:
5′-GAGCTGTTTCATGTTTATCCTCCCCAGCTC-3′
SEQ ID NO. 12:
5′-GGTCGAGCTGTTTCATGTTTATCCTCCCCAGCTCGACC-3′
SEQ ID NO. 13:
5′-CTGGTCGAGCTGTTTCATGTTTATCCTCCCCAGCTCGACCAG-3′
SEQ ID NO. 14:
5′-AGCTGGTCGAGCTGTTTCATGTTTATCCTCCCCAGCTCGACCAGCT-3′
SEQ ID NO. 15:
5′-GAAGCTGGTCGAGCTGTTTCATGTTTATCCTCCCCAGCTCGACCAGCTTC-3′
SEQ ID NO. 16:
5′-TACACTTTCATGTTTATCCTCCCTAAACATGAAAGTGTA-3′
SEQ ID NO. 17:
5′-ATTGTTTTCATGTTTATCCTCCCTAAACATGAAAACAAT-3′
SEQ ID NO. 18:
5′-CTTTCATGTTTATCCTCCCTAAACATGAAAG-3′
SEQ ID NO. 19:
5′-ACTTTCATGTTTATCCTCCCTAAACATGAAAGT-3′
SEQ ID NO. 20:
5′-CACTTTCATGTTTATCCTCCCTAAACATGAAAGTG-3′
SEQ ID NO. 21:
5′-ACACTTTCATGTTTATCCTCCCTAAACATGAAAGTGT-3′
SEQ ID NO. 22:
5′-CTACACTTTCATGTTTATCCTCCCTAAACATGAAAGTGTAG-3′
SEQ ID NO. 23:
S′-ACTACACTTTCATGTTTATCCTCCCTAAACATGAAAGTGTAGT-3′
SEQ ID NO. 24:
5′-GACTACACTTTCATGTTTATCCTCCCTAAACATGAAAGTGTAGTC-3′
SEQ ID NO. 25:
5′-AGACTACACTTTCATGTTTATCCTCCCTAAACATGAAAGTGTAGTCT-3′
SEQ ID NO. 26:
5′-CAGACTACACTTTCATGTTTATCCTCCCTAAACATGAAAGTGTAGTCTG-3′
SEQ ID NO. 27:
5′-TAAACATGAAACATGTTTCATGTTTCATGTTTATCCTCCC-3′
SEQ ID NO. 28:
5′-CACACTTTCATGTTTATCCTCCCTAAACATGAAAGTGT-3′
SEQ ID NO. 29:
5′-CACACTTTCATGTTTATCCTCCCTAAACATGAAAGTGTN-3′
SEQ ID NO. 30-
5′-TTTCATGTTTATCCTCCCACATGAAA-3′
SEQ ID NO. 31-
5′-TTTCATGTTTATCCTCCCAACATGAAA-3′
SEQ ID NO. 32-
5′-TTTCATGTTTATCCTCCCTAAACATGAAA-3′
SEQ ID NO. 33-
5′-CAGACTACATTTTCATGTTTATCCTCCCTAAACATGAAAATGTAGTCTG-3′
SEQ ID NO. 34-
5′-CAGACTACGTTTTCATGTTTATCCTCCCTAAACATGAAAACGTAGTCTG-3′
SEQ ID NO. 35-
5′-CAGACTATGTTTTCATGTTTATCCTCCCTAAACATGAAAACATAGTCTG-3′
SEQ ID NO. 36-
5′-CAGACTTTGTTTTCATGTTTATCCTCCCTAAACATGAAAACAAAGTCTG-3′
SEQ ID NO. 37-
5′-CAGACATTGTTTTCATGTTTATCCTCCCTAAACATGAAAACAATGTCTG~3′
SEQ ID NO. 38-
5′-CAGATATTGTTTTCATGTTTATCCTCCCTAAACATGAAAACAATATCTG-3′
SEQ ID NO. 39-
5′-CAGTTATTGTTTTCATGTTTATCCTCCCTAAACATGAAAACAATAACTG-3′
SEQ ID NO. 40-
5′-CAATTATTGTTTTCATGTTTATCCTCCCTAAACATGAAAACAATAATTG-3′
SEQ ID NO. 41-
5′-CGATTATTGTTTTCATGTTTATCCTCCCTAAACATGAAAACAATAATCG-3′
SEQ ID NO. 42-
5′-AGATTATTGTTTTCATGTTTATCCTCCCTAAACATGAAAACAATAATCT-3′
SEQ ID NO. 43-
5′-TGTACGATTTTCATGTTTATCCTCCCGAAAATCGTACA-3′
SEQ ID NO. 44-
S′-GGGAGGATAAACATGAAA-3′
SEQ ID NO. 45-
5′-TTTCATGTTTATCCTCCC-3′
SEQ ID NO. 46-
5′-TTTCATGTTTATCCTCCC-3′
SEQ ID NO. 47-
5′-GGGTGGATAAACATGAAA-3′
SEQ ID NO. 48-
5′-GGGAGGTTAAACATGAAA-3′
SEQ ID NO. 49-
5′-GGGAGGATATACATGAAA-3′
SEQ ID NO. 50-
5′-GGGAGGATAAACATCAAA-3′
SEQ ID NO. 51-
5′-AGGGAGGATTTTCATGTTTATCCTCCCT-3′
SEQ ID NO. 52-
5′-ATTTTGGGAGGATAAACATGAAAACAATAATCTTAGCTAGCCTTA-3′
SEQ ID NO. 53-
5′-ATTTTGGGAGGATAAACATGAAAACAATAATCTTAGCTAGCCTTA~3′
SEQ ID NO. 54-
3′-TACAAATA AAAACCCTCCTATTTGTA-5′
SEQ ID NO. 55-
S′-CTTTCATGTTTATCCTCCCTAAACATGAAAG-3′
SEQ ID NO. 56-
5′-ACTTTCATGTTTATCCTCCCTAAACATGAAAGT-3′
SEQ ID NO. 57-
5′-CACTTTCATGTTTATCCTCCCTAAACATGAAAGTG-3′

1. A detection probe comprising:
a nucleic acid sequence comprising a fingerloop structure comprising:
an antisense sequence region located in a loop and in one strand of a stem loop of the fingerloop structure, wherein the antisense sequence region is complementary to a target nucleic acid sequence; and
a nonsense sequence region located at the base of the stem loop of the fingerloop structure;
wherein:
the antisense sequence region of the loop comprises at least 3 nucleotides;
the antisense sequence region of the stem loop comprises at least 6 nucleotides; and
the nonsense sequence region of the stem loop comprises at least 1 nucleotide.

2. The detection probe of claim 1, wherein the one strand of the stem loop is a descending strand of the fingerloop structure.

3. The detection probe of claim 1, wherein the one strand of the stem loop is an ascending strand of the fingerloop structure.

4. The detection probe of claim 3, wherein the nucleic acid sequence further comprises a fluorescent nucleoside at a 3′ end of the ascending strand of the fingerloop structure.

5. The detection probe of claim 2, wherein the nucleic acid sequence further comprises a fluorescent nucleoside at a 5′ end of the descending strand of the fingerloop structure.

6. The detection probe of claim 2, wherein the nonsense sequence is located before the antisense sequence region at a 3′ end of a descending strand or after the antisense sequence region at a 5′ end of a ascending strand.

7. The detection probe of claim 4, wherein the fluorescent nucleoside is thienoguanosine.

8. The detection probe of claim 1, wherein the detection probe has a ΔG of −30 kcal/mol or less.

9. The detection probe of claim 1, wherein the nucleic acid comprises DNA, DNA derivatives, such as LNAs, PNAs, 2′-Fluoro RNAs, or other commonly used synthetic nucleic acid analogs.

10. The detection probe of claim 1, wherein the fingerloop structure diminishes base pairing to a mismatched target nucleic acid.

11. The detection probe of claim 1, wherein the detection probe is a Northern blot probe.

12. The detection probe of claim 1, wherein the detection probe is a Southern blot probe.

13. The detection probe of claim 1, wherein the antisense sequence region comprises from 10 to 40 nucleotides.

14. The detection probe of claim 1, wherein the antisense sequence region of the loop comprises from 3 to 20 nucleotides.

15. The detection probe of claim 1, wherein the antisense sequence region of the stem loop comprises from 6 to 20 nucleotides in one strand of a stem loop.

16. The detection probe of claim 1, wherein the nonsense sequence region of the stem loop comprises from 1 to 10 nucleotides in one strand of a stem loop.

17. A method for detecting a target nucleic acid sequence, the method comprising:
providing a nucleic acid sequence;
contacting the nucleic acid sequence with a detection probe of any one of claims 1-16;
assessing a change in fluorescence intensity;
wherein an increase in fluorescence intensity indicates binding of the detection probe to a target nucleic acid sequence.

18. The method of claim 17, wherein the detection probe diminishes base pairing to a mismatched target nucleic acid.

19. The detection probe of claim 17, wherein the fingerloop structure provides increased detection specificity of the target nucleic acid sequence.

20. A method of tuning a fingerloop stability, the method comprising:
adding a nonsense nucleic acid sequence to a nucleic acid sequence comprising a fingerloop structure comprising:
an antisense sequence region located in a loop and in one strand of a stem loop of the fingerloop structure, wherein the antisense sequence region is complementary to a target nucleic acid sequence, wherein:
the antisense sequence region of the loop comprises at least 3 nucleotides;
the antisense sequence region of the stem loop comprises at least 6 nucleotides in one strand of a stem loop;
wherein the nonsense nucleic acid sequence is located at the base of the stem loop of the fingerloop structure; and
wherein the nonsense sequence region of the stem loop comprises at least 1 nucleotide in one strand of a stem loop.

21.-49. (canceled)


SEQUENCE LISTING (57 sequences)

SEQ ID NO: 1 - RNA, 28 nt, synthetic construct
  source 1..28: mol_type = other RNA
atttcatgtttatcctcccacatgaaat

SEQ ID NO: 2 - RNA, 29 nt, synthetic construct
  source 1..29: mol_type = other RNA
atttcatgtttatcctcccaacatgaaat

SEQ ID NO: 3 - RNA, 30 nt, synthetic construct
  source 1..30: mol_type = other RNA
atttcatgtttatcctccctaacatgaaat

SEQ ID NO: 4 - RNA, 30 nt, synthetic construct
  source 1..30: mol_type = other RNA
ctttcatgtttatcctccctaacatgaaag

SEQ ID NO: 5 - RNA, 32 nt, synthetic construct
  source 1..32: mol_type = other RNA
actttcatgtttatcctccctaacatgaaagt

SEQ ID NO: 6 - RNA, 34 nt, synthetic construct
  source 1..34: mol_type = other RNA
cactttcatgtttatcctccctaacatgaaagtg

SEQ ID NO: 7 - DNA, 32 bp, synthetic construct
  source 1..32: mol_type = other DNA
cacactttcatgtttatcctcccgaaagtgtg

SEQ ID NO: 8 - DNA, 36 bp, synthetic construct
  source 1..36: mol_type = other DNA
aacacacactttcatgtttatcctcccgtgtgtgtt

SEQ ID NO: 9 - DNA, 26 bp, synthetic construct
  source 1..26: mol_type = other DNA
gctgtttcatgtttatcctccccagc

SEQ ID NO: 10 - DNA, 34 bp, synthetic construct
  source 1..34: mol_type = other DNA
tcgagctgtttcatgtttatcctccccagctcga

SEQ ID NO: 11 - DNA, 30 bp, synthetic construct
  source 1..30: mol_type = other DNA
gagctgtttcatgtttatcctccccagctc

SEQ ID NO: 12 - DNA, 38 bp, synthetic construct
  source 1..38: mol_type = other DNA
ggtcgagctgtttcatgtttatcctccccagctcgacc

SEQ ID NO: 13 - DNA, 42 bp, synthetic construct
  source 1..42: mol_type = other DNA
ctggtcgagctgtttcatgtttatcctccccagctcgaccag

SEQ ID NO: 14 - DNA, 46 bp, synthetic construct
  source 1..46: mol_type = other DNA
agctggtcgagctgtttcatgtttatcctccccagctcgaccagct

SEQ ID NO: 15 - DNA, 50 bp, synthetic construct
  source 1..50: mol_type = other DNA
gaagctggtcgagctgtttcatgtttatcctccccagctcgaccagcttc

SEQ ID NO: 16 - DNA, 39 bp, synthetic construct
  source 1..39: mol_type = other DNA
tacactttcatgtttatcctccctaaacatgaaagtgta

SEQ ID NO: 17 - DNA, 39 bp, synthetic construct
  source 1..39: mol_type = other DNA
attgttttcatgtttatcctccctaaacatgaaaacaat

SEQ ID NO: 18 - DNA, 31 bp, synthetic construct
  source 1..31: mol_type = other DNA
ctttcatgtttatcctccctaaacatgaaag

SEQ ID NO: 19 - DNA, 33 bp, synthetic construct
  source 1..33: mol_type = other DNA
actttcatgtttatcctccctaaacatgaaagt

SEQ ID NO: 20 - DNA, 35 bp, synthetic construct
  source 1..35: mol_type = other DNA
cactttcatgtttatcctccctaaacatgaaagtg

SEQ ID NO: 21 - DNA, 37 bp, synthetic construct
  source 1..37: mol_type = other DNA
acactttcatgtttatcctccctaaacatgaaagtgt

SEQ ID NO: 22 - DNA, 41 bp, synthetic construct
  source 1..41: mol_type = other DNA
ctacactttcatgtttatcctccctaaacatgaaagtgtag

SEQ ID NO: 23 - DNA, 43 bp, synthetic construct
  source 1..43: mol_type = other DNA
actacactttcatgtttatcctccctaaacatgaaagtgtagt

SEQ ID NO: 24 - DNA, 45 bp, synthetic construct
  source 1..45: mol_type = other DNA
gactacactttcatgtttatcctccctaaacatgaaagtgtagtc

SEQ ID NO: 25 - DNA, 47 bp, synthetic construct
  source 1..47: mol_type = other DNA
agactacactttcatgtttatcctccctaaacatgaaagtgtagtct

SEQ ID NO: 26 - DNA, 49 bp, synthetic construct
  source 1..49: mol_type = other DNA
cagactacactttcatgtttatcctccctaaacatgaaagtgtagtctg

SEQ ID NO: 27 - DNA, 40 bp, synthetic construct
  source 1..40: mol_type = other DNA
taaacatgaaacatgtttcatgtttcatgtttatcctccc

SEQ ID NO: 28 - DNA, 38 bp, synthetic construct
  source 1..38: mol_type = other DNA
cacactttcatgtttatcctccctaaacatgaaagtgt

SEQ ID NO: 29 - DNA, 39 bp, synthetic construct
  source 1..39: mol_type = other DNA
  modified_base 39: mod_base = OTHER; note = thienoguanosine
cacactttcatgtttatcctccctaaacatgaaagtgtn

SEQ ID NO: 30 - DNA, 26 bp, synthetic construct
  source 1..26: mol_type = other DNA
tttcatgtttatcctcccacatgaaa

SEQ ID NO: 31 - DNA, 27 bp, synthetic construct
  source 1..27: mol_type = other DNA
tttcatgtttatcctcccaacatgaaa

SEQ ID NO: 32 - DNA, 29 bp, synthetic construct
  source 1..29: mol_type = other DNA
tttcatgtttatcctccctaaacatgaaa

SEQ ID NO: 33 - DNA, 49 bp, synthetic construct
  source 1..49: mol_type = other DNA
cagactacattttcatgtttatcctccctaaacatgaaaatgtagtctg

SEQ ID NO: 34 - DNA, 49 bp, synthetic construct
  source 1..49: mol_type = other DNA
cagactacgttttcatgtttatcctccctaaacatgaaaacgtagtctg

SEQ ID NO: 35 - DNA, 49 bp, synthetic construct
  source 1..49: mol_type = other DNA
cagactatgttttcatgtttatcctccctaaacatgaaaacatagtctg

SEQ ID NO: 36 - DNA, 49 bp, synthetic construct
  source 1..49: mol_type = other DNA
cagactttgttttcatgtttatcctccctaaacatgaaaacaaagtctg

SEQ ID NO: 37 - DNA, 49 bp, synthetic construct
  source 1..49: mol_type = other DNA
cagacattgttttcatgtttatcctccctaaacatgaaaacaatgtctg

SEQ ID NO: 38 - DNA, 49 bp, synthetic construct
  source 1..49: mol_type = other DNA
cagatattgttttcatgtttatcctccctaaacatgaaaacaatatctg

SEQ ID NO: 39 - DNA, 49 bp, synthetic construct
  source 1..49: mol_type = other DNA
cagttattgttttcatgtttatcctccctaaacatgaaaacaataactg

SEQ ID NO: 40 - DNA, 49 bp, synthetic construct
  source 1..49: mol_type = other DNA
caattattgttttcatgtttatcctccctaaacatgaaaacaataattg

SEQ ID NO: 41 - DNA, 49 bp, synthetic construct
  source 1..49: mol_type = other DNA
cgattattgttttcatgtttatcctccctaaacatgaaaacaataatcg

SEQ ID NO: 42 - DNA, 49 bp, synthetic construct
  source 1..49: mol_type = other DNA
agattattgttttcatgtttatcctccctaaacatgaaaacaataatct

SEQ ID NO: 43 - DNA, 38 bp, synthetic construct
  source 1..38: mol_type = other DNA
tgtacgattttcatgtttatcctcccgaaaatcgtaca

SEQ ID NO: 44 - DNA, 18 bp, synthetic construct
  source 1..18: mol_type = other DNA
gggaggataaacatgaaa

SEQ ID NO: 45 - DNA, 18 bp, synthetic construct
  source 1..18: mol_type = other DNA
tttcatgtttatcctccc

SEQ ID NO: 46 - DNA, 18 bp, synthetic construct
  source 1..18: mol_type = other DNA
tttcatgtttatcctccc

SEQ ID NO: 47 - DNA, 18 bp, synthetic construct
  source 1..18: mol_type = other DNA
gggtggataaacatgaaa

SEQ ID NO: 48 - DNA, 18 bp, synthetic construct
  source 1..18: mol_type = other DNA
gggaggttaaacatgaaa

SEQ ID NO: 49 - DNA, 18 bp, synthetic construct
  source 1..18: mol_type = other DNA
gggaggatatacatgaaa

SEQ ID NO: 50 - DNA, 18 bp, synthetic construct
  source 1..18: mol_type = other DNA
gggaggataaacatcaaa

SEQ ID NO: 51 - DNA, 28 bp, synthetic construct
  source 1..28: mol_type = other DNA
agggaggattttcatgtttatcctccct

SEQ ID NO: 52 - DNA, 45 bp, synthetic construct
  source 1..45: mol_type = other DNA
attttgggaggataaacatgaaaacaataatcttagctagcctta

SEQ ID NO: 53 - DNA, 45 bp, synthetic construct; identical to SEQ ID NO: 52
  source 1..45: mol_type = other DNA

SEQ ID NO: 54 - DNA, 26 bp, synthetic construct
  source 1..26: mol_type = other DNA
atgtttatcctcccaaaaataaacat

SEQ ID NO: 55 - DNA, 31 bp, synthetic construct; identical to SEQ ID NO: 18
  source 1..31: mol_type = other DNA

SEQ ID NO: 56 - DNA, 33 bp, synthetic construct; identical to SEQ ID NO: 19
  source 1..33: mol_type = other DNA

SEQ ID NO: 57 - DNA, 35 bp, synthetic construct; identical to SEQ ID NO: 20
  source 1..35: mol_type = other DNA